(19) United States
(12) Patent Application Publication
(10) Pub. No.: US 2005/0226946 A1
(43) Pub. Date: Oct. 13, 2005

(54) Composition obtained from barley shochu stillage and having effects of inhibiting the onset of alcoholic liver injury and healing it as well as favorable taste, and processing for producing the same

(75) Inventors: Toshiro Omori, Oita (JP); Naoki Takeshima, Oita (JP); Kei Hayashi, Oita (JP); Yasufumi Umemoto, Oita (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignees: SANWA SHURUI CO., LTD, Usa-shi (JP); BARLEY FERMEHTATION TECHNOLOGIES, INC., Usa-shi (JP)

(21) Appl. No.: 10/511,725

(22) PCT Filed: Apr. 17, 2003
(86) PCT No.: PCT/JP03/04893

§ 371 Date: Oct. 19, 2004

(30) Foreign Application Priority Data
Apr. 19, 2002 (JP) 2002-117431
Apr. 19, 2002 (JP) 2002-117435
Aug. 29, 2002 (JP) 2002-250991
Aug. 29, 2002 (JP) 2002-250992

(51) Int. Cl. A61K 35/78; A61K 38/06; A61K 38/08

(52) U.S. Cl. 424/750; 514/17; 514/18

(57) ABSTRACT

A composition having effects of inhibiting the onset of alcoholic liver injury and healing it which comprises an unadsorbed fraction obtained by subjecting a barley shochu stillage obtained as the by-product in the production of shochu from barley to solid/liquid separation and then subjecting the liquid fraction thus obtained to a separation treatment by adsorption. The unadsorbed fraction contains plural peptides having an average chain length of from 3.0 to 5.0. These peptides are composed of from 24 to 38% of glutamic acid, from 4 to 20% of glycine, from 5 to 10% of aspartic acid, from 4 to 9% of proline and from 4 to 8% of serine, referring to the total amino acid content originating in these peptides as 100%. A food composition comprising the above unadsorbed fraction and having a favorable taste is also disclosed.

### TECHNICAL FIELD

The present invention relates to a composition having an outstanding activity of inhibiting the onset of alcoholic hepatopathy and an outstanding activity of healing it as well as an excellent palatability, the composition comprising an unadsorbed fraction which is formed by subjecting a barley shochu stillage to solid-liquid separation to obtain a liquid fraction and subjecting the liquid fraction to a separation treatment by adsorption using a synthetic adsorbent, and a process for producing the same.

More specifically, the invention relates to a composition having an outstanding activity of inhibiting the onset of alcoholic hepatopathy and an outstanding activity of healing it, the composition comprising an unadsorbed fraction which is formed by subjecting a barley shochu stillage byproduced in the production of shochu using a barley as a raw material to solid-liquid separation to obtain a liquid fraction and subjecting the liquid fraction to a separation treatment by adsorption using a synthetic adsorbent, and a process for producing the same. Further, the invention relates to a food composition having an excellent palatability and comprising the unadsorbed fraction, and a process for producing the same.

The alcoholic hepatopathy referred to in the invention includes alcoholic hepatitis, alcoholic fatty liver and alcoholic hyperlipemia induced by excess intake of alcohols.

### BACKGROUND ART

With the increase in consumption amount of alcoholic beverages in recent years, the number of persons suffering from the alcoholic hepatopathy has been increased, and the alcoholic hepatopathy has been recognized as one of life-style related diseases. Specific diseases of the alcoholic hepatopathy can include alcoholic fatty liver, alcoholic hepatitis, alcoholic liver fibrosis, alcoholic liver cirrhosis, alcoholic hyperlipemia induced by alcoholic fatty liver, and the like. It is known that when the alcoholic fatty liver becomes chronic, it is shifted to the alcoholic liver fibrosis in which fibers are generated around hepatocytes, and that when the amount of blood entered into hepatocytes is reduced in the alcoholic liver fibrosis to decrease an ability of synthesizing proteins of the liver or an ability of decomposing toxic substances, the liver becomes gradually hard which leads to the alcoholic liver cirrhosis.

By the way, regarding the hepatopathy preventing activity of a barley shochu stillage byproduced in the production of barley shochu (hereinafter simply referred to as “abarley shochu stillage”), the following matters are known. That is, it has been reported that a barley shochu stillage has an activity of inhibiting accumulation of lipid in a rat's liver caused by administration of orotic acid [refer to Nihon Eiyo-Shokuryo Gakkai Sokai Koen Yoshishu, Vol. 53, 53(1999)] (hereinafter referred to as “document 1”). Further, it has been reported that an activity of inhibiting fatty liver which is provided by this barley shochu stillage is strong in comparison to that of vine lees or beer lees and this activity is not found at all in a sweet potato shochu stillage and is very low in a rice shochu stillage so that this activity is peculiar to the barley shochu stillage only [refer to Nihon Jozo Kyokaishi, Vol. 94, No. 9, 768 (1999)] (hereinafter referred to as “document 2”). Still further, it has been reported that an activity of inhibiting the onset of D-galactosamine-induced hepatopathy known to show the similar condition to viral hepatopathy is observed in a liquid fraction obtained by subjecting a barley shochu stillage to centrifugation [Nihon Jozo Kyokaishi, Vol. 95, No. 9, 706 (2000)] (hereinafter referred to as “document 3”).

As stated above, documents 1 to 3 describe that the barley shochu stillage or the liquid fraction obtained by subjecting the barley shochu stillage to solid-liquid separation has the activity of inhibiting the onset of orotic acid-induced hepatopathy or D-galactosamine-induced hepatopathy. However, they do not even suggest whether or not it has an activity of inhibiting alcoholic hepatopathy or healing it.

JP-A-2001-145472 (hereinafter referred to as “document 4”) describes that a composition comprising an ethanol-insoluble fraction containing an organic acid, protein and hemicellulose and formed by subjecting a barley shochu stillage to solid-liquid separation to obtain a liquid fraction, adding an alkali to the liquid fraction to collect an alkali-soluble fraction, neutralizing the alkali-soluble fraction with an acid to obtain a neutral soluble fraction and adding ethanol to the neutral soluble fraction has an activity of inhibiting the onset of orotic acid-induced hepatopathy in an experiment using rats.

Thus, document 4 describes that the ethanol-insoluble fraction has an activity of inhibiting the onset of orotic acid-induced hepatopathy. However, it does not even suggest whether or not the ethanol-insoluble fraction has an activity of inhibiting the onset of alcoholic hepatopathy or an activity of healing it.

As stated above, an example of obtaining a fraction having an activity of inhibiting the onset of alcoholic hepatopathy and an activity of healing it from a barley shochu stillage has been to date entirely unknown.

By the way, the orotic acid-induced hepatopathy is known to be hepatopathy in which synthesis of fat in the liver is accelerated with orotic acid and migration of fat from the liver into blood is inhibited to thereby induce the fat liver. The D-galactosamine-induced hepatopathy is known to be hepatopathy in which necrosis of hepatocytes is accelerated with D-galactosamine to thereby induce hepatitis.

Meanwhile, the alcoholic hepatopathy includes, as noted earlier, alcoholic hepatitis, alcoholic fatty liver and alcoholic hyperlipemia induced by excess intake of alcohol, and it is objectively differentiated from the orotic acid-induced hepatopathy and the D-galactosamine-induced hepatopathy. That is, the alcoholic fat liver is known to be a fat liver in which neutral fat is accumulated in the liver by accelerating migration of a fatty acid from a fat tissue to the liver with ethanol to accelerate synthesis of fatty acid or neutral fat in the liver, inhibiting decomposition of fatty acid in the liver and the like. The alcoholic hepatitis is known to be hepatitis which is induced such that acetaldehyde or acetic acid, a metabolite of ethanol or active oxygen generated in producing the same damages hepatocytes. The alcoholic hyperlipemia is known to be triggered such that excess neutral fat accumulated in the liver is released to blood in large quantities as a secretory very low density lipoprotein (VLDL). In such an alcoholic hepatopathy, it is known that lesion of hepatitis such as balloon-like swelling or necrosis of hepatocytes, or a fatty liver comprising hepatocytes containing large fatty drops is progressed mainly on the terminal hepatic vein peripheral region of the hepatic lobule. Incidentally, the liver is an assembly of a large number of the hepatic lobules each having a diameter of 1 mm in which the hepatic lobule partitioned by an interlobular connective tissue functions as one unit.

Accordingly, in view of the causative sequence of such hepatopathies, the alcoholic hepatopathy is objectively differentiated from the orotic acid-induced hepatopathy and the D-galactosamine-induced hepatopathy. Even though some ingredient is known to have an activity of inhibiting the onset of orotic acid-induced hepatopathy or D-galactosamine-induced hepatopathy or an activity of healing it, it can never be expected easily whether or not the very ingredient has also an activity of inhibiting the alcoholic hepatopathy or an activity of healing it.

JP-A-6-98750 (hereinafter referred to as “document 5”) describes a seasoning comprising a polished concentrate of a shochu stillage obtained by heating at from 80 to 95° C. a residue formed by distilling a fermentation product of cereals and/or potatoes and separating shochu, then removing a solid matter, subjecting the resulting solution to adsorption with an adsorbent such as activated carbon and then concentrating the product to a Brix degree of from 25 to 50. There is nothing in document 5 to describe whether or not the polished concentrate has any pharmacological action.

Document 5 describes that the polished concentrate which can be used as a seasoning for imparting a good taste to a food without any putrefaction or malodor even in the long-term storage is obtained by the heat treatment step of “heating at from 80 to 95° C. a stillage formed by distilling a fermentation product of cereals and/or potatoes and separating shochu”. However, in document 5, there is a problem that since amino acids and the like having a palatability and contained in the shochu stillage are degenerated in the heat treatment step which is conducted at the high temperature of from 80 to 95° C., the palatability is notably decreased to reduce a value as a seasoning. Further, a degree of coloration of the polished concentrate is more increased by such a heat treatment step. Accordingly, the polished concentrate described in document 5 is problematic in that the use as a seasoning is limited. There is nothing in document 5 to describe whether or not the polished concentrate has any pharmacological action.

### SUMMARY OF THE INVENTION

The invention has been made under the foregoing circumstances of the prior art.

That is, in view of the fact that documents 1 to 3 describe that the barley shochu stillage or the liquid fraction obtained by subjecting the barley shochu stillage to solid-liquid separation (these are hereinafter referred to as “the liquid fraction of the barley shochu stillage”) has the activity of inhibiting the onset of D-galactosamine-induced hepatopathy or orotic acid-induced hepatopathy, the present inventors have assiduously conducted investigations through experiments for the purpose of clarifying whether or not the liquid fraction of the barley shochu stillage has the activity of inhibiting the onset of alcoholic hepatopathy. As a result, it has been found that the liquid fraction of the barley shochu stillage has slightly the activity of inhibiting the onset of alcoholic hepatopathy but it is not suggestive of the actual use as a drug for positively inhibiting the onset of alcoholic hepatopathy.

By the way, the three of the present inventors have found with the other two that the adsorbed fraction obtained by subjecting the liquid fraction of the barley shochu stillage to the separation treatment by adsorption using the synthetic adsorbent has the activity of inhibiting the onset of orotic acid-induced fatty liver and D-galactosamine-induced hepatopathy, whereas the unadsorbed fraction byproduced in the separation treatment by adsorption does not have the activity of inhibiting the onset of the orotic acid-induced fatty liver and the D-galactosamine-induced hepatopathy (already applied for patent as Japanese Patent Application 2002-56929). Accordingly, since the unadsorbed fraction does not have the pharmacological action, it has been discarded as a waste. Nevertheless, the present inventors have examined, through experiments, the activity of inhibiting the onset of the alcoholic hepatopathy and the activity of healing it using the unadsorbed fraction which has been discarded as a waste. Consequently, they have found that the unadsorbed fraction has surprisingly the very strong activity of inhibiting the onset of the alcoholic hepatopathy and the strong activity of healing the alcoholic hepatopathy. From this fact, the unadsorbed fraction has been found to be useful as a drug.

Further, the present inventors have conducted investigations on the unadsorbed fraction in view of the palatability. Consequently, it has been found that the unadsorbed fraction formed by subjecting the barley shochu stillage to solid-liquid separation to obtain a liquid fraction and subjecting the liquid fraction to a separation treatment by adsorption using a synthetic adsorbent has, in comparison to the polished concentrate of document5 (polished concentrate obtained by the method of document 5 using the barley shochu stillage as a raw material), an outstanding palatability with a rough taste extremely reduced and is extremely low in degree of coloration and it can therefore be used as a seasoning advantageously. Still further, the present inventors have conducted investigations, through experiments, as to whether or not the polished concentrate described in document 5 has the activity of inhibiting the onset of the alcoholic hepatopathy. As a result, it has been found that the polished concentrate has slightly the activity of inhibiting the onset of the alcoholic hepatopathy but its extent is quite low and it is not suggestive of the actual use as a drug for positively inhibiting the onset of the alcoholic hepatopathy. Incidentally, when the activity of inhibiting the onset of the alcoholic hepatopathy is quite low as in the polished concentrate, the activity of healing the alcoholic hepatopathy is naturally quite low. Therefore, it is not considered that only the activity of healing the same is increased. In view of the foregoing, it has been found that the activity of inhibiting the onset of the alcoholic hepatopathy and the activity of healing it which are provided by the unadsorbed fraction are extremely great and cannot be attained by the polished concentrate described in document 5.

Under these circumstances, the invention has been completed.

It is an object of the invention to provide a composition having an activity of inhibiting the onset of alcoholic hepatopathy and an activity of healing it, the composition comprising an unadsorbed fraction which is formed by subjecting a barley shochu stillage to solid-liquid separation to obtain a liquid fraction and subjecting the liquid fraction to a separation treatment by adsorption using a synthetic adsorbent, and a process for producing the same.

Another object of the invention is to provide a food composition having an activity of inhibiting the onset of alcoholic hepatopathy and an activity of healing it as well as an excellent palatability, the composition comprising an unadsorbed fraction which is formed by subjecting a barley shochu stillage to solid-liquid separation to obtain a liquid fraction and subjecting the liquid fraction to a separation treatment by adsorption using a synthetic adsorbent, and a process for producing the same.

The unadsorbed fraction in the invention is characterized in that it basically contains plural peptides having an average chain length of from 3.0 to 5.0, and these peptides comprise from 24 to 38% of glutamic acid, from 4 to 20% of glycine, from 5 to 10% of aspartic acid, from 4 to 9% of proline and from 4 to 8% of serine in terms of an amino acid composition ratio when the total content of amino acids derived from the peptides is defined as 100%.

By the way, the ethanol-insoluble fraction described in document 4 is obtained by a method which is entirely different from the method of obtaining the unadsorbed fraction by subjecting the liquid fraction of the barley shochu stillage (liquid fraction obtained by subjecting the barley shochu stillage to liquid-solid separation) to the separation treatment by adsorption using the synthetic adsorbent in the invention. That is, the ethanol-insoluble fraction described in document 4 is formed by adding an alkali to the liquid fraction of the barley shochu stillage to obtain an alkali-soluble fraction, neutralizing the alkali-soluble fraction with an acid to obtain a neutral soluble fraction and adding ethanol to the neutral soluble fraction for precipitation. The ethanol-insoluble fraction contains 28±3% by weight of hemicellulose as one of main components, and this hemicellulose has a saccharide composition of from 60 to 70% by weight of xylose. Meanwhile, the unabsorbed fraction of the invention contains the plural peptides having the average chain length of from 3.0 to 5.0, which are not described at all in document 4. Although the unadsorbed fraction of the invention contains from 15 to 25% by weight of polysaccharides (which will be described later), the saccharide composition of the polysaccharides is no doubt different from that of the hemicellulose. Accordingly, the ethanol-insoluble fraction described in document 4 is clearly different from the unadsorbed fraction of the invention.

The experiments that the present inventors performed during the completion of the invention are described below.

Experiment 1

In view of the fact that documents 1 to 3 describe that the liquid fraction of the barley shochu stillage has the activity of inhibiting the onset of the D-galactosamine-induced hepatopathy and the orotic acid-induced fatty liver as described above, the present inventors performed the following experiment using a liquid fraction (A) of the barley shochu stillage prepared for clarifying whether or not the liquid fraction of the barley shochu stillage has also the activity of inhibiting the onset of the alcoholic hepatopathy, and they made assiduous studies.

That is, an ethanol-containing liquid feed was given to 24 three-week-old Wistar-strain male rats (Nippon SLC) for 6 days while gradually raising the ethanol content (3%→4%→5%), and these rats were then divided into two groups, a control group and a test group, each group consisting of 12 rats. At this time, the 24 rats were divided so as not to cause a statistically significant difference in distribution of the average weight of the rats in each of the groups. The rats of the control group were bred by giving a 5% ethanol-containing liquid feed for 4 weeks, and the rats of the test group by giving for 4 weeks a liquid feed comprising the 5% ethanol-containing liquid feed and 1% of a freeze-dried powder (A′) of the liquid fraction (A) of the barley shochu stillage, respectively. Apart from the control group and the test group, an untreated group consisting of the 12 three-week-old Wistar-strain male rats was provided. The rats of the untreated group was bred by giving for 4 weeks an ethanol-free liquid feed containing a mixture of maltose and dextrin in equal amounts instead of 5% ethanol for making the intake calory equal to that of the other two groups. In this case, the feeding amount (intake calory) of each liquid feed per day was limited to 70 ml (70 kcal) in the three groups. On the final day of the experiment (week 4 after the start-up of the test), blood was sampled from the abdominal aorta of each bred rat, and the liver was extracted. After the serum was separated from the sampled blood, serum total cholesterol, serum HDL-cholesterol, serum LDL-cholesterol, serumtriglyceride, serumphospholipid, serum free fatty acid and serum ALT (GPT) were measured. With respect to the extracted liver, liver weight, liver total cholesterol, liver triglyceride and liver phospholipid were measured. The results obtained were shown in terms of mean value±standard error (SEM), and the statistical treatment was conducted in the following manner. That is, the comparison between the untreated group and the control group was analyzed using the Student's test method, and the comparison between the control group and the test group was then analyzed using the Tukey-Kramer method. In each analysis, the evaluation was conducted on condition that the level of significance of less than 0.05% was defined as significant. Further, after hepatocytes collected from the extracted liver were subjected to HE staining, morphological observation of the hepatocytes was conducted with 200 x magnification using a biological microscope BX51 manufactured by Olympus Optical Co., Ltd.

Consequently, it has been found that the control group is, in comparison to the untreated group, significantly increased in serum total cholesterol concentration, serum HDL-cholesterol concentration, serum LDL-cholesterol concentration, serum triglyceride concentration and serum phospholipid concentration to induce the alcoholic hyperlipemia. Further, it has been found that the control group is, in comparison to the untreated group, significantly increased in liver triglyceride concentration and liver phospholipid concentration to induce the alcoholic fatty liver. Still further, it has been found that the control group is, in comparison to the untreated group, significantly increased in blood ALT (GPT) concentration and the necrosis of hepatocytes and the balloon-like swelling in the terminal hepatic vein peripheral region of the hepatic lobule are notably found in the observation of the hepatocytes through the biological microscope to induce the alcoholic hepatitis. Meanwhile, the test group has shown, in comparison to the control group, a tendency of inhibiting the increase in serum LDL-cholesterol concentration, serum triglyceride concentration and liver triglyceride concentration and a tendency of slightly decreasing the necrosis of hepatocytes and the balloon-like swelling in the terminal hepatic vein peripheral region of the hepatic lobule in the observation of the hepatocytes through the biological microscope.

The foregoing results have revealed that the freeze-dried powder (A′) of the liquid fraction (A) of the barley shochu stillage is not suggestive of the actual use as a drug for positively inhibiting the onset of alcoholic hepatopathy.

Experiment 2

Next, in order to clarify, through the following experiment, what fraction obtained from the liquid fraction of the barley shochu stillage contributes to the activity of inhibiting the onset of alcoholic hepatopathy, the present inventors performed the experiment using a desorbed fraction (B) obtained by subjecting the liquid fraction of the barley shochu fraction to a separation treatment by adsorption using a synthetic adsorbent and eluting the resulting adsorbed fraction with an alkali, and they conducted assiduous studies.

That is, an ethanol-containing liquid feed was given to 24 three-week-old Wistar-strain male rats (Nippon SLC) for 6 days while gradually raising the ethanol content (3%→4%→+5%), and these rats were then divided into two groups, a control group and a test group, each group consisting of 12 rats. At this time, the 24 rats were divided so as not to cause a statistically significant difference in distribution of the average weight of the rats in each of the groups. The rats of the control group were bred by giving a 5% ethanol-containing liquid feed for 4 weeks, and the rats of the test group by giving for 4 weeks a liquid feed comprising the 5% ethanol-containing liquid feed and 1% of a freeze-dried powder (B′) of the desorbed fraction (B), respectively. Apart from the control group and the test group, an untreated group consisting of the 12 three-week-old Wistar-strain male rats was provided. The rats of the untreated group was bred by giving for 4 weeks an ethanol-free liquid feed containing a mixture of maltose and dextrin in equal amounts instead of 5% ethanol for making the intake calory equal to that of the other two groups. In this case, the feeding amount (intake calory) of each liquid feed per day was limited to 70 ml (70 kcal) in the three groups. On the final day of the experiment (week 4 after the start-up of the test), blood was sampled from the abdominal aorta of each bred rat, and the liver was extracted. After the serum was separated from the sampled blood, serum total cholesterol, serum HDL-cholesterol, serum LDL-cholesterol, serum triglyceride, serum phospholipid, serum free fatty acid and serum ALT (GPT) were measured. With respect to the extracted liver, liver weight, liver total cholesterol, liver triglyceride and liver phospholipid were measured. The results were shown in terms of mean value±standard error (SEM), and the statistical treatment was conducted in the following manner. That is, the comparison between the untreated group and the control group was analyzed using the Student's test method, and the comparison between the control group and the test group was then analyzed using the Tukey-Kramer method. In each analysis, the evaluation was conducted on condition that the level of significance of less than 0.05% was defined as significant. Further, after hepatocytes collected from the extracted liver was subjected to HE staining, morphological observation of the hepatocytes was conducted with 200× magnification using a biological microscope BX51 manufactured by Olympus Optical Co., Ltd.

Consequently, it has been found that the control group is, in comparison to the untreated group, significantly increased in serum total cholesterol concentration, serum HDL-cholesterol concentration, serum LDL-cholesterol concentration, serum triglyceride concentration and serum phospholipid concentration to induce the alcoholic hyperlipemia. Further, it has been found that the control group is, in comparison to the untreated group, significantly increased in liver triglyceride concentration and liver phospholipid concentration to induce the alcoholic fatty liver. Still further, it has been found that the control group is, in comparison to the untreated group, significantly increased in blood ALT (GPT) concentration and the necrosis of hepatocytes and the balloon-like swelling in the terminal hepatic vein peripheral region of the hepatic lobule are notably found in the observation of the hepatocytes through the biological microscope to induce the alcoholic hepatitis. Meanwhile, the test group has shown, in comparison to the control group, a tendency of inhibiting the increase in serum triglyceride concentration of the rats, but the increase in liver triglyceride concentration has not been inhibited at all, and the necrosis of hepatocytes and the balloon-like swelling in the terminal hepatic vein peripheral region of the hepatic lobule have been notably found also in the observation of the hepatocytes through the biological microscope. That is, the freeze-dried powder (B′) of the desorbed fraction (B) has slightly shown a tendency of inhibiting induction of the alcoholic hyperlipemia, but has not shown at all a tendency of inhibiting induction of the alcoholic fatty liver and the alcoholic hepatitis.

The foregoing results has revealed that the freeze-dried powder (′B) of the desorbed fraction (B) is substantially free from the activity of inhibiting the onset of the alcoholic hepatopathy.

Experiment 3

The present inventors have thus presumed that the unadsorbed fraction formed by subjecting the liquid fraction of the barley shochu stillage to a separation treatment by adsorption using a synthetic adsorbent positively shows the activity of inhibiting the onset of the alcoholic hepatopathy. They performed the following experiment using an unadsorbed fraction (C) formed by subjecting the liquid fraction of the barley shochu stillage to a separation treatment by adsorption using a synthetic adsorbent, and they conducted assiduous studies.

That is, an ethanol-containing liquid feed was given to 24 three-week-old Wistar-strain male rats (Nippon SLC) for 6 days while gradually raising the ethanol content (3%→4%→5%), and these rats were then divided into two groups, a control group and a test group, each group consisting of 12 rats. At this time, the 24 rats were divided so as not to cause a statistically significant difference in distribution of the average weight of the rats in each of the groups. The rats of the control group were bred by giving a 5% ethanol-containing liquid feed for 4 weeks, and the rats of the test group by giving for 4 weeks a liquid feed comprising the 5% ethanol-containing liquid feed and 1% of a freeze-dried powder (C′) of the desorbed fraction (C), respectively. Apart from the control group and the test group, an untreated group consisting of the 12 three-week-old Wistar-strain male rats was provided. The rats of the untreated group was bred by giving for 4 weeks an ethanol-free liquid feed containing a mixture of maltose and dextrin in equal amounts instead of 5% ethanol for making the intake calory equal to that of the other two groups. In this case, the feeding amount (intake calory) of each liquid feed per day was limited to 70 ml (70 kcal) in the three groups. On the final day of the experiment (week 4 after the start-up of the test), blood was sampled from the abdominal aorta of each bred rat, and the liver was extracted. After the serum was separated from the sampled blood, serum total cholesterol, serum HDL-cholesterol, serum LDL-cholesterol, serum triglyceride, serum phospholipid, serum free fatty acid and serum ALT (GPT) were measured. With respect to the extracted liver, liver weight, liver total cholesterol, liver triglyceride and liver phospholipid were measured. The results were shown in terms of mean value±standard error (SEM), and the statistical treatment was conducted in the following manner. That is, the comparison between the untreated group and the control group was analyzed using the Student's test method, and the comparison between the control group and the test group was then analyzed using the Tukey-Kramer method. In each analysis, the evaluation was conducted on condition that the level of significance of less than 0.05% was defined as significant. Further, after hepatocytes collected from the extracted liver were subjected to HE staining, morphological observation of the hepatocytes was conducted with 200× magnification using a biological microscope BX51 manufactured by Olympus Optical Co., Ltd.

Consequently, it has been found that the control group is, in comparison to the untreated group, significantly increased in serum total cholesterol concentration, serum HDL-cholesterol concentration, serum LDL-cholesterol concentration, serum triglyceride concentration and serum phospholipid concentration to induce the alcoholic hyperlipemia. Further, it has been found that the control group is, in comparison to the untreated group, significantly increased in liver triglyceride concentration and liver phospholipid concentration to induce the alcoholic fatty liver. Still further, it has been found that the control group is, in comparison to the untreated group, significantly increased in blood ALT (GPT) concentration and the necrosis of hepatocytes and the balloon-like swelling in the terminal hepatic vein peripheral region of the hepatic lobule are notably found in the observation of the hepatocytes through the biological microscope to induce the alcoholic hepatitis. Meanwhile, the test group has significantly inhibited the increase in serum LDL-cholesterol concentration, serum triglyceride concentration and liver triglyceride concentration of the rats, and the necrosis of hepatocytes and the balloon-like swelling in the terminal hepatic vein peripheral region of the hepatic lobule have been little found in the observation of the hepatocytes through the biological microscope.

The foregoing results of the experiment have revealed that the freeze-dried powder (C′) of the unadsorbed fraction (C) has the marked activity of inhibiting the onset of the alcoholic hepatopathy.

Experiment 4

The present inventors assiduously conducted investigations on the unadsorbed fraction (C) in view of the palatability. Consequently, they have found that the unadsorbed fraction formed by subjecting the barley shochu stillage to solid-liquid separation to obtain a liquid fraction and subjecting the liquid fraction to a separation treatment by adsorption can be a seasoning which has an outstanding palatability with a rough taste extremely reduced and has quite a low degree of coloration, in comparison to the seasoning made of the polished concentrate obtained by using the barley shochu stillage as a raw material directly without any treatment as described in document 5.

Experiment 5

As stated above, document 5 never describes the pharmacological action of the polished concentrate. However, in view of the fact that it describes that the polished concentrate is obtained from the residue formed by distilling a fermentation product of cereals and/or potatoes and separating shochu, a polished product (D) was formed by subjecting the barley shochu stillage as a raw material to the same process as in document 5, and was examined through the experiment for clarifying whether or not the polished concentrate (D) has the activity of inhibiting the onset of the alcoholic hepatopathy.

That is, an ethanol-containing liquid feed was given to 24 three-week-old Wistar-strain male rats (Nippon SLC) for 6 days while gradually raising the ethanol content (3%→4%→5%), and these rats were then divided into two groups, a control group and a test group, each group consisting of 12 rats. At this time, the 24 rats were divided so as not to cause a statistically significant difference in distribution of the average weight of the rats in each of the groups. The rats of the control group were bred by giving a 5% ethanol-containing liquid feed for 4 weeks, and the rats of the test group by giving for 4 weeks a liquid feed comprising the 5% ethanol-containing liquid feed and 1% of a freeze-dried powder (D¹) of the polished concentrate (D), respectively. Apart from the control group and the test group, an untreated group consisting of the 12 three-week-old Wistar-strain male rats was provided. The rats of the untreated group were bred by giving for 4 weeks an ethanol-free liquid feed containing a mixture of maltose and dextrin in equal amounts instead of 5% ethanol for making the intake calory equal to that of the other two groups. In this case, the feeding amount (intake calory) of each liquid feed per day was limited to 70 ml (70 kcal) in the three groups. On the final day of the experiment (week 4 after the start-up of the test), blood was sampled from the abdominal aorta of each bred rat, and the liver was extracted. After the serum was separated from the sampled blood, serum total cholesterol, serum HDL-cholesterol, serum LDL-cholesterol, serum triglyceride, serum phospholipid, serum free fatty acid and serum ALT were measured. With respect to the extracted liver, liver weight, liver total cholesterol, liver triglyceride and liver phospholipid were measured. The results obtained were shown in terms of mean value±standard error (SEM), and the statistical treatment was conducted in the following manner. That is, the comparison between the untreated group and the control group was analyzed using the Student's test method, and the comparison between the control group and the test group was then analyzed using the Tukey-Kramer method. In each analysis, the evaluation was conducted on condition that the level of significance of less than 0.05% was defined as significant. Further, after hepatocytes collected from the extracted liver were subjected to HE staining, morphological observation of the hepatocytes was conducted with 200× magnification using a biological microscope BX51 manufactured by Olympus Optical Co., Ltd.

Consequently, it has been found that the control group is, in comparison to the untreated group, significantly increased in serum total cholesterol concentration, serum HDL-cholesterol concentration, serum LDL-cholesterol concentration, serum triglyceride concentration and serum phospholipid concentration to induce the alcoholic hyperlipemia. Further, it has been found that the control group is, in comparison to the untreated group, significantly increased in liver triglyceride concentration and liver phospholipid concentration to induce the alcoholic fatty liver. Still further, it has been found that the control group is, in comparison to the untreated group, significantly increased in blood ALT (GPT) concentration and the necrosis of hepatocytes and the balloon-like swelling in the terminal hepatic vein peripheral region of the hepatic lobule are notably found in the observation of the hepatocytes through the biological microscope to induce the alcoholic hepatitis. Meanwhile, it has been found that the test group shows, in comparison to the control group, a tendency of inhibiting the increase in serum LDL-cholesterol concentration, serum triglyceride concentration and liver triglyceride concentration and a tendency of slightly decreasing the necrosis of hepatocytes and the balloon-like swelling in the terminal hepatic vein peripheral region of the hepatic lobule in the observation of the hepatocytes through the biological microscope to provide the slight activity of inhibiting the onset of the alcoholic hepatopathy. However, it has been found that the activity of inhibiting the onset of the alcoholic hepatopathy which activity is provided by the freeze-dried powder (D′) of the polished concentrate (D) is substantially the same as the activity of inhibiting the onset which activity is provided by the liquid fraction of the barley shochu stillage as described in documents 2 to 4 and stated in Experiment 1, meaning that it is not suggestive of the actual use as a drug for positively inhibiting the onset of the alcoholic hepatopathy. Accordingly, it has been found that the activity of inhibiting the onset of the alcoholic hepatopathy which activity is provided by the polished concentrate (D) is much lower than the activity of inhibiting the onset which activity is provided by the unadsorbed fraction (C) described in Experiment 3 and it is not suggestive of the actual use as a drug for positively inhibiting the onset of the alcoholic hepatopathy.

The foregoing results have revealed that the ingredient contributing to the activity of inhibiting the onset of the alcoholic hepatopathy which activity is provided by the barley shochu stillage is present in a state quite fractionated in the unadsorbed fraction formed by subjecting the liquid fraction of the barley shochu stillage to the separation treatment by adsorption using the synthetic adsorbent. Further, it has been found that the ingredient contributing to the activity of inhibiting the onset of the alcoholic hepatopathy cannot be obtained by the process described in document 5.

Experiment 6

Thus, the present inventors performed the following experiment for clarifying whether or not the freeze-dried powder (A′) of the liquid fraction (A) of the barley shochu stillage described in Experiment 1 which powder has been found to be unsuggestive of the actual use as a drug for positively inhibiting the onset of the alcoholic hepatopathy and the freeze-dried powder (C′) of the unadsorbed fraction (C) described in Experiment 3 which powder has been found to have the marked activity of inhibiting the onset of the alcoholic hepatopathy possess the activity of healing the alcoholic hepatopathy already triggered.

That is, an ethanol-containing liquid feed was given to 30 seven-week-old Wistar-strain male rats (Nippon Charles River) for 6 days for breeding while gradually raising the ethanol content (3%→4%→5%), and these rats were successively bred with a 5% ethanol-containing liquid feed for 4 weeks. On week 4, blood was sampled from each of the rats. The plasma was separated to measure a serum lipid. The rats were divided into three groups, a control group, a test group 1 and a test group 2, each group consisting of 10 rats. At this time, the 30 rats were divided so as not to cause a statistically significant difference in distribution of the average weight of the rats in each of the groups. The rats of the control group were bred by giving for 2 weeks an ethanol-free liquid feed containing a mixture of maltose and dextrin in equal amounts instead of 5% ethanol for making the intake calory equal to that of the ethanol-containing liquid feed administration groups. The rats of the test group 1 were bred by giving for 2 weeks a liquid feed comprising the ethanol-free liquid feed and 1% of the freeze-dried powder (A′) of the liquid fraction (A). The rats of the test group 2 were bred by giving for 2 weeks a liquid feed containing the ethanol-free liquid feed and 1% of the freeze-dried powder (C′) of the unadsorbed fraction (C). Further, apart from the control group, the test group 1 and the test group 2, an untreated group consisting of the 10 seven-week-old Wistar-strain male rats was provided. The rats of the untreated group were bred by giving the ethanol-free liquid feed for 6 weeks. In this case, the feeding amount (intake calory) of each liquid feed per day was limited to 70 ml (70 kcal) in the four groups. With respect to all of the four groups, on the final day of the experiment (week 6 after the start-up of the experiment), blood was sampled from the abdominal aorta of each bred rat, and the liver was extracted. After the serum was separated from the sampled blood, serum total cholesterol, serum LDL-cholesterol, serumtriglyceride, serumphospholipid, serum ALT (GPT) and serum AST (GOT) were measured. With respect to the extracted liver, liver weight, liver total cholesterol, liver triglyceride and liver phospholipid were measured. The results were shown in terms of mean value±standard error (SEM), and the statistical treatment was conducted in the following manner. That is, the comparison between the untreated group and the control group was analyzed using the Student's test method, and the comparison between the control group and the A and B groups was then analyzed using the Tukey-Kramer method. In each analysis, the evaluation was conducted on condition that the level of significance of less than 0.05% was defined as significant. Further, after hepatocytes collected from the extracted liver was subjected to HE staining, morphological observation of the hepatocytes was conducted with 200× magnification using a biological microscope BX51 manufactured by Olympus Optical Co., Ltd.

Consequently, in the control group, serum total cholesterol concentration, serum LDL-cholesterol concentration and liver triglyceride concentration increased by breeding with the ethanol-containing liquid feed have been slightly decreased, and the extents approximate to the normal values of the respective concentrations have been quite small. Further, the test group 1 has shown, in comparison to the control group, a tendency of decreasing serum triglyceride concentration, serum total cholesterol concentration, serum phospholipid concentration, serum LDL-cholesterol concentration, serum ALT (GTP) concentration, serum AST (GOT) concentration and liver total cholesterol concentration, but the extents approximate to the normal values of the respective concentrations have been small. Meanwhile, in the test group 2, serum triglyceride concentration, serum total cholesterol concentration, serum phospholipid concentration, serum LDL-cholesterol concentration, serum ALT concentration, serum AST concentration and liver total cholesterol concentration have been values which are significantly lower than those in the control group, namely values which are substantially equal to the normal values of the respective concentrations. The necrosis of hepatocytes and the balloon-like swelling in the terminal hepatic vein peripheral region of the hepatic lobule have been little found in the observation of the hepatocytes through the biological microscope. That is, it has been found that the activity of healing the alcoholic hepatopathy which activity is provided by the freeze-dried powder (A′) of the liquid fraction (A) is not suggestive of the actual use as a drug for positively healing the alcoholic hepatopathy, whereas the freeze-dried powder (C′) of the unadsorbed fraction (C) outstandingly shows the activity of healing the alcoholic hepatopathy.

The results of Experiments 1 to 6 have revealed the following facts. That is, the unadsorbed fraction formed by subjecting the barley shochu stillage byproduced in the production of shochu from a barley as a raw material to solid-liquid separation to obtain a liquid fraction and subjecting the liquid fraction to a separation treatment by adsorption using a synthetic adsorbent is quite strong in activity of inhibiting the onset of the alcoholic hepatopathy and activity of healing it. Further, the unadsorbed fraction has quite an excellent palatability. The liquid fraction formed by subjecting the barley shochu stillage by produced in the production of shochu from a barley as a raw material to solid-liquid separation contains the ingredient contributing to the inhibition of the onset of the alcoholic hepatopathy and the healing of the alcoholic hepatopathy, and this ingredient is contained by being fractionated in the unabsorbed fraction formed by subjecting the liquid fraction to the separation treatment by adsorption using the synthetic adsorbent.

The present inventors analyzed the unadsorbed fraction by the analytical method to be described later in Examples. Consequently, it has been found that the unadsorbed fraction contains plural peptides having an average chain length of from 3.0 to 5.0, these peptides comprise from 24 to 38% of glutamic acid, from 4 to 20% of glycine, from 5 to 10% of aspartic acid, from 4 to 9% of proline and from 4 to 8% of serine in terms of an amino acid composition ratio when the total content of amino acids derived from the peptides is defined as 100%, and the total content of amino acids derived from the peptides is from 8 to 14% by weight. Further, it has been found that the unadsorbed fraction contains free amino acids, free saccharides, polysaccharides and organic acids, and that specifically the unadsorbed fraction contains from 4 to 12% by weight of the free amino acids, from 5 to 10% by weight of the free saccharides, from 15 to 25% by weight of the polysaccharides and from 2 to 8% by weight of the organic acids. It has been found that the free amino acids comprise amino acids, namely from 20 to 28% of proline, from 11 to 18% of alanine, from l to 17% of leucine, from 10 to 17% of arginine and from 13 to 20% of glutamic acid, that the free saccharides have a saccharide composition comprising from 2 to 6% by weight of glucose, from 0.5 to 5% by weight of xylose and from 0.5 to 3% by weight of arabinose, and that the polysaccharides have a saccharide composition comprising from 6 to 16% by weight of glucose, from 3 to 12% by weight of xylose and from 0.5 to 4% by weight of arabinose. It has been found that the organic acids include citric acid, malic acid, succinic acid and lactic acid. Moreover, it has been found that when the unadsorbed fraction is freeze-dried, a light yellow condition is provided. In addition, the unadsorbed fraction contains from 15 to 25% by weight of the foregoing polysaccharides, it has been presumed that some of the peptides are bound to such polysaccharides.

The invention is based on these facts which have been found.

### DESCRIPTION OF THE INVENTION AND THE PREFERRED EMBODIMENTS THEREOF

The invention provides a composition having the outstanding activity of inhibiting the onset of alcoholic hepatopathy and the outstanding activity of healing it and comprising an unadsorbed fraction which is formed by subjecting a barley shochu stillage byproduced in the production of shochu from a barley as a raw material to solid-liquid separation to obtain a liquid fraction and subjecting the liquid fraction to a separation treatment by adsorption using a synthetic adsorbent, in which the unadsorbed fraction contains plural peptides having an average chain length of from 3.0 to 5.0 and these peptides comprise from 24 to 38% of glutamic acid, from 4 to 20% of glycine, from 5 to 10% of aspartic acid, from 4 to 9% of proline and from 4 to 8% of serine in terms of an amino acid composition ratio when the total content of amino acids derived from the peptides is defined as 100%, and a process for producing the same.

Further, the invention provides a food composition having an outstanding activity of inhibiting the onset of alcoholic hepatopathy and an outstanding activity of healing it as well as an excellent palatability, the composition comprising an unadsorbed fraction which is formed by subjecting a barley shochu stillage byproduced in the production of shochu from a barley as a raw material to solid-liquid separation to obtain a liquid fraction and subjecting the liquid fraction to a separation treatment by adsorption using a synthetic adsorbent, in which the unadsorbed fraction contains plural peptides having an average chain length of from 3.0 to 5.0 and these peptides comprise from 24 to 38% of glutamic acid, from 4 to 20% of glycine, from 5 to 10% of aspartic acid, from 4 to 9% of proline and from 4 to 8% of serine in terms of an amino acid composition ratio when the total content of amino acids derived from the peptides is defined as 100%, and a process for producing the same.

The preferred embodiments of the invention are described below. However, the invention is not limited to these.

The compositions (the composition having a strong activity of inhibiting the onset of alcoholic hepatopathy and a strong activity of healing it, and the food composition having a strong activity of inhibiting the onset of alcoholic hepatopathy and a strong activity of healing it as well as an excellent palatability) can be produced by a process comprising a first step of subjecting a barley shochu stillage byproduced in the production of a distilled liquor using a barley to solid-liquid separation and a second step of subjecting the liquid fraction to a separation treatment by adsorption using a synthetic adsorbent.

The barley shochu stillage byproduced in the production of shochu from a barley as a raw material, which stillage is used in performing the process of the invention, and the steps of the process are described in detail below.

The barley shochu stillage used in the invention means a substance which is byproduced as a distillation residue when producing a barley koji and a steamed barley from a barley or a polished barley as a raw material, saccharifying a starch contained in the resulting barley koji and steamed barley with a koji and/or an enzyme, further subjecting the resulting product to alcohol fermentation with a yeast to obtain a mature mash and distilling the mature mash with a distillation device of vacuum distillation, normal pressure distillation or the like, namely a stillage of barley shochu. Further, a shochu stillage byproduced when using a barley as a part of a raw material in the production of rice shochu, sweet potato shochu and buckwheat shochu is also included in the barley shochu stillage used in the invention.

In the production of the barley shochu stillage in the invention, the barley koji used to produce the barley shochu may be produced under conditions of koji production which are employed in the usual production of barley shochu. As the koji mold to be used, a white-koji mold (Aspergillus kawachii) which is generally used in the production of barley shochu is preferable. Or molds belonging to the genus Aspergillus, such as a black-kojimold (Aspergillus awamori) used in the production of awamori and a yellow-koji mold (Aspergillus oryzae) used in the production of sake or the like, can also be used. As the yeast used in the production of the barley shochu, various yeasts for brewing of shochu generally used in the production of shochu can be used.

In the invention, the first step of subjecting the barley shochu stillage obtained during the distillation step in the production of the barley shochu to solid-liquid separation to obtain the liquid fraction is performed for the purpose of removing from the barley shochu stillage a water-insoluble fermentation residue derived from the barley or the barley koji as a raw material to obtain the liquid fraction. The solid-liquid separation in the first step can be performed by a solid-liquid separation method of a screw press system or a roller press system. Alternatively, it can be performed by a method in which a preliminary solid-liquid separation treatment is conducted using a solid-liquid separator of a filtration-compression system and a main solid-liquid separation treatment is conducted using a centrifugal separator, a diatomaceous earth filter, a ceramic filter, a filtration compressor or the like.

The second step of subjecting the liquid fraction obtained in the first step to a separation treatment by adsorption using a synthetic adsorbent to obtain an unadsorbed fraction is performed for the purpose of fractionating with the synthetic adsorbent an ingredient contained in the liquid fraction and involved in the activity of inhibiting the onset of alcoholic hepatopathy and the activity of healing it. Preferable specific examples of the synthetic adsorbent used in the second step can include Amberlite XAD-4, Amberlite XAD-16, Amberlite XAD-1180 and Amberlite XAD-2000 manufactured by Organo K.K., aromatic (or also referred to as styrenic) synthetic adsorbents such as Sepabeads SP850 and Diaion HP20 manufactured by Mitsubishi Chemical Corp., and methacrylic (or also referred to as acrylic) synthetic adsorbents such as Amberlite XAD-7 manufactured by Organo K.K. and Diaion HP2MG manufactured by Mitsubishi Chemical Corp. Besides these adsorbents, aromatic modified synthetic adsorbents such as Sepabeads SP207 manufactured by Mitsubishi Chemical Corp. can be used as required.

The thus-obtained unadsorbed fraction can be used, either directly or in the form of a dry powder by being subjected to freeze-drying or the like, as a drug composition having the activity of inhibiting the onset of alcoholic hepatopathy and the activity of healing it or as a food composition having the activity of inhibiting the onset of alcoholic hepatopathy and the activity of healing it as well as the quite excellent palatability, especially preferably as a seasoning.

The invention is described more specifically below by referring to Examples. However, the invention is not limited at all by these Examples.

Barley shochu was produced for use in the following Examples. As a raw material, a barley (70% polished) was used.

[Production of a Barley Koji]

A barley was caused to absorb water by 40% (w/w), steamed for 40 minutes, and then allowed to cool to 40° C. A mold starter (white-koji mold) was inoculated in an amount of 1 kg per ton of the barley, and maintained at 38° C. and RH 95% for 24 hours and at 32° C. and RH 92% for 20 hours to produce a barley koji.

[Production of a Steamed Barley]

A barley was caused to absorb water by 40% (w/w), steamed for 40 minutes, and then allowed to cool to 40° C. to produce a steamed barley.

[Production of Barley Shochu and a Barley Shochu Stillage]

In the primary work, 3.6 kiloliters of water and 1 kg (wet weight) of a culture strain of a shochu yeast as a yeast were added to the above-produced barley koji (3 tons as abarley) produced above in [Production of a barley koji] to obtain a primary mash, and the resulting primary mash was subjected to fermentation (first fermentation) for 5 days. Then, in the second work, 11.4 kiloliters of water and the steamed barley (7 tons as a barley) produced above in [Production of a steamed barley] were added to the primary mash after the first fermentation, and the mixture was subjected to fermentation (second fermentation) for 11 days. The fermentation temperature was set at 25° C. in both of the first and second works. The secondary mash after the second fermentation was subjected to single distillation in a usual manner to obtain 10 kiloliters of barley shochu and 15 kiloliters of a barley shochu stillage. The resulting barley shochu stillage was used in the following Example 1, Comparative Example 1, Comparative Example 2 and Reference Example 1.

### EXAMPLE 1

The barley shochu stillage obtained above in [Production of barley shochu and a barley shochu stillage] was centrifuged under conditions of 8,000 rpm and 10 minutes to form a liquid fraction of the barley shochu stillage, and 25 L of the liquid fraction and 10 L of deionized water were passed in this order through a column (resin volume 10 L) filled with a synthetic adsorbent Amberlite XAD-16 manufactured by Organo K.K. to obtain an unadsorbed fraction comprising a bypassed solution showing an unadsorbability to the synthetic adsorbent of the column. The resulting unadsorbed fraction was freeze-dried with a vacuum freeze-dryer to obtain 1,200 g of a freeze-dried product. The resulting freeze-dried product was pulverized to give a light yellow powder.

### COMPARATIVE EXAMPLE 1

The powder of the liquid fraction of the barley shochu stillage described in documents 1 to 3 which stillage is known to have the activity of inhibiting the onset of D-galactosamine-induced hepatopathy and orotic acid-induced hepatopathy was formed by the following method. That is, the barley shochu stillage obtained above in [Production of barley shochu and a barley shochu stillage] was centrifuged under conditions of 8,000 rpm and 10 minutes to form a liquid fraction. 25 L of the resulting liquid fraction was freeze-dried with a vacuum freeze-dryer to give 1,500 g of a freeze-dried product. The resulting freeze-dried product was pulverized to give a light brown powder.

### COMPARATIVE EXAMPLE 2

The powder comprising the polished concentrate of document 5 was obtained by the following method. That is, 10 L of the barley shochu stillage obtained above in [Production of barley shochu and a barley shochu stillage] was heated at 90° C., maintained for 30 minutes while being stirred, then cooled to 50° C., and subjected to solid-liquid separation using a solid-liquid separator of a filter press system to obtain a liquid fraction. To the liquid fraction were added 1% by weight of carbon particles and 0.3% by weight of perlite. The mixture was maintained at 50° C., stirred, and then filtered through a super carbon filter to obtain 9L of a filtrate. 9L of this filtrate was freeze-dried with a vacuum freeze-dryer to obtain 567 g of a freeze-dried product. The resulting freeze-dried product was pulverized to give a brown powder.

### REFERENCE EXAMPLE 1

A powder of an adsorbed fraction formed by subjecting a liquid fraction resulting from solid-liquid separation of a barley shochu stillage to a separation treatment by adsorption using a synthetic adsorbent was obtained by the following method. That is, the barley shochu stillage obtained above in [Production of barley shochu and a barley shochu stillage] was centrifuged under conditions of 8,000 rpm and 10 minutes to form a liquid fraction, and 25 L of the liquid fraction and 10 L of deionized water were passed in this order through a column (resin volume 10 L) filled with a synthetic adsorbent Amberlite XAD-16 manufactured by Organo K.K. to remove a bypassed solution comprising an unadsorbed fraction showing an unadsorbability to the synthetic adsorbent of the column. Then, 10 L of a 1 (wt/vol) % sodium hydroxide solution and 10 L of deionized water were passed through the column in this order to obtain 20 L of an elute containing an adsorbed fraction adsorbed on the synthetic adsorbent of the column. 20L of this elute was passed through a column (resin volume 10 L) filled with a strongly acidic cation exchange resin IR-120B manufactured by Organo K.K. for a desalting treatment. The resulting liquid was freeze-dried with a vacuum freeze-dryer to obtain 270 g of a freeze-dried product with sodium ions removed. The resulting freeze-dried product was pulverized to give a brown powder.

The freeze-dried powder obtained in Example 1 and the freeze-dried powders obtained in Comparative Examples 1 and 2 and Reference Example 1 were subjected to the following Test Example 1 to evaluate the activity of inhibiting the onset of alcoholic hepatopathy.

### TEST EXAMPLE 1

The following test was conducted to confirm that the composition of the invention (freeze-dried powder obtained in Example 1) has an outstanding activity of inhibiting the onset of alcoholic hepatopathy.

That is, an ethanol-containing liquid feed was given to 60 three-week-old Wistar-strain male rats (Nippon SLC) for 6 days while gradually raising the ethanol content (3%→4%→5%), and these rats were then divided into five groups, a control group, an A group, a B group, a C group and a D group, each group consisting of 12 rats. At this time, the 60 rats were divided so as not to cause a statistically significant difference in distribution of the average weight of the rats in each of the groups. The rats of the control group were bred by giving a 5% ethanol-containing liquid feed for 4 weeks. The rats of the A group were bred by giving for 4 weeks a liquid feed comprising a 5% ethanol-containing liquid feed and 1% of the freeze-dried powder obtained in Example 1. The rats of the B group were bred by giving for 4 weeks a liquid feed comprising the 5% ethanol-containing liquid feed and 1% of the freeze-dried powder obtained in Comparative Example 1. The rats of the C group were bred by giving for 4 weeks a liquid feed comprising a 5% ethanol-containing liquid feed and 1% of the freeze-dried powder obtained in Comparative Example 2. The rats of the D group were bred by giving for 4 weeks a liquid feed comprising a 5% ethanol-containing liquid feed and 1% of the freeze-dried powder obtained in Reference Example 1. Apart from the control group, the A group, the B group, the C group and the D group, an untreated group consisting of the 12 three-week-old Wistar-strain male rats was provided. The rats of the untreated group were bred by giving for 4 weeks an ethanol-free liquid feed containing a mixture of maltose and dextrin in equal amounts instead of 5% ethanol for making the intake calory equal to that of the other five groups. In this case, the feeding amount (intake calory) of each liquid feed per day was limited to 70 ml (70 kcal) in the six groups. On the final day of the experiment (week 4 after the start-up of the test), blood was sampled from the abdominal aorta of each bred rat, and the liver was extracted. After the serum was separated from the sampled blood, serum total cholesterol, serum HDL-cholesterol, serum LDL-cholesterol, serumtriglyceride, serumphospholipid, serum-free fatty acid and serum ALT were measured. With respect to the extracted liver, liver weight, liver total cholesterol, liver triglyceride and liver phospholipid were measured. The results obtained were shown in terms of mean value±standard error (SEM), and the statistical treatment was conducted in the following manner. That is, the comparison between the untreated group and the control group was analyzed using the Student's test method, and the comparison between the control group and the A to D groups was then analyzed using the Tukey-Kramer method. In each analysis, the evaluation was conducted on condition that the level of significance of less than 0.05% was defined as significant. Further, after hepatocytes collected from the extracted liver were subjected to HE staining, morphological observation of the hepatocytes was conducted with 200× magnification using a biological microscope BX51 manufactured by Olympus Optical Co., Ltd.

The results of measuring serum total cholesterol, serum HDL-cholesterol, serum LDL-cholesterol, serum triglyceride, serum phospholipid, serum free fatty acid and serum ALT are shown in Table 1, and the results of measuring liver weight, liver total cholesterol, liver triglyceride and liver phospholipid are shown in Table 2.

Considerations Based on the Results Shown in Tables 1 and 2:

The following facts have been found on the basis of the results shown in Tables 1 and 2. That is, it has been found that the control group is, in comparison to the untreated group, significantly increased in serum total cholesterol concentration, serum HDL-cholesterol concentration, serum LDL-cholesterol concentration, serum triglyceride concentration and serum phospholipid concentration to induce the alcoholic hyperlipemia. Further, it has been found that the control group is, in comparison to the untreated group, significantly increased in liver triglyceride concentration and liver phospholipid concentration to induce the alcoholic fatty liver. Still further, it has been found that the control group is, in comparison to the untreated group, significantly increased in blood ALT (GPT) concentration and the necrosis of hepatocytes and the balloon-like swelling in the terminal hepatic vein peripheral region of the hepatic lobule are notably found in the observation of the hepatocytes through the biological microscope to induce the alcoholic hepatitis. Mean while, in the A group, the increase in serum LDL-cholesterol concentration, serum triglyceride concentration and liver triglyceride concentration has been significantly inhibited, and the necrosis of hepatocytes and the balloon-like swelling in the terminal hepatic vein peripheral region of the hepatic lobule, which are specifically observed in the alcoholic hepatopathy, have been little found in the observation of the hepatocytes through the biological microscope. The B group and the C group have shown a tendency of inhibiting the increase in serum LDL-cholesterol concentration, serum triglyceride concentration and liver triglyceride concentration and also a tendency of slightly decreasing the necrosis of hepatocytes and the balloon-like swelling in the terminal hepatic vein peripheral region of the hepatic lobule in the observation of the hepatocytes through the biological microscope. The D group has shown a tendency of inhibiting the increase in serum triglyceride concentration. However, the increase in liver triglyceride has not been inhibited at all, and the necrosis of hepatocytes and the balloon-like swelling in the terminal hepatic vein peripheral region of the hepatic lobule, which are specifically observed in the alcoholic hepatopathy, have been notably found in the observation of the hepatocytes through the biological microscope.

The foregoing results have revealed that the freeze-dried powders obtained in Comparative Examples 1 and 2 show the slight activity of inhibiting the onset of alcoholic hepatopathy which is not suggestive of the actual use as a drug for positively inhibiting the onset of alcoholic hepatopathy and the freeze-dried powder obtained in Reference Example 1 does not inhibit at all the induction of alcoholic hepatopathy. Meanwhile, it has been found that the freeze-dried powder obtained in Example 1 (composition of the invention) markedly inhibits the induction of the alcoholic hepatopathy, showing the strong activity of inhibiting the onset of alcoholic hepatopathy. That is, it has been found that the freeze-dried powder obtained in Example 1 has a markedly strong activity of inhibiting the onset of alcoholic hepatopathy and is useful as a drug.

### TEST EXAMPLE 2

With respect to the freeze-dried powder obtained in Example 1 (composition of the invention) and the freeze-dried powder obtained in Comparative Example 1, the activity of healing the alcoholic hepatopathy was evaluated as described below.

That is, rats afflicted with the alcoholic hepatopathy by breeding with the ethanol-containing liquid feed for 4 weeks were bred using the freeze-dried powder obtained in Example 1 and the freeze-dried powder obtained in Comparative Example 1 to evaluate the activity of healing the alcoholic hepatopathy.

An ethanol-containing liquid feed was given to 30 seven-week-old Wister-strain male rats (Nippon Charles River) for 6 days for breeding while gradually raising the ethanol content (3%→4%→5%), and these rats were successively bred with a 5% ethanol-containing liquid feed for 4 weeks. After 4 weeks of the breeding, blood was sampled from each of the rats, and the plasma was separated to measure serum lipid. These rats were then divided into three groups, a control group, an A group and a B group, each group consisting of 10 rats. At this time, the 30 rats were divided so as not to cause a statistically significant difference in distribution of the average weight of the rats in each of the groups. The rats of the control group were bred by giving for 2 weeks an ethanol-free liquid feed containing a mixture of maltose and dextrin in equal amounts instead of 5% ethanol for making the intake calory equal to that of the ethanol-containing liquid feed administration groups. The rats of the A group were bred by giving for 2 weeks a liquid feed comprising an ethanol-free liquid feed and 1% of the freeze-dried powder obtained in Example 1. The rats of the B group were bred by giving for 2 weeks a liquid feed comprising an ethanol-free liquid feed and 1% of the freeze-dried powder obtained in Comparative Example 1. Apart from the control group, the A group and the B group, an untreated group consisting of the 10 seven-week-old Wistar-strain male rats was provided. The rats of the untreated group were bred by giving an ethanol-free liquid feed for 6 weeks. In this case, the feeding amount (intake calory) of each liquid feed per day was limited to 70 ml (70 kcal) in the four groups. With respect to all of the four groups, on the final day of the breeding (week 6 after the start-up of the breeding), blood was sampled from the abdominal aorta of each bred rat, and the liver was extracted. After the serum was separated from the sampled blood, serum total cholesterol, serum LDL-cholesterol, serum triglyceride, serum phospholipid, serum ALT (GPT) and serum AST (GOT) were measured. With respect to the extracted liver, liver weight, liver total cholesterol, liver triglyceride and liver phospholipid were measured. The results obtained were shown in terms of mean value±standard error (SEM), and the statistical treatment was conducted in the following manner. That is, the comparison between the untreated group and the control group was analyzed using the Student's test method, and the comparison between the control group and the A and B groups was then analyzed using the Tukey-Kramer method. In each analysis, the evaluation was conducted on condition that the level of significance of less than 0.05% was defined as significant. Further, after hepatocytes collected from the extracted liver were subjected to HE staining, morphological observation of the hepatocytes was conducted with 200× magnification using a biological microscope BX51 manufactured by Olympus Optical Co., Ltd.

The results of measuring serum total cholesterol, serum LDL-cholesterol, serumtriglyceride, serumphospholipid, serum ALT and serum AST are shown in Table 3, and the results of measuring liver weight, liver total cholesterol, liver triglyceride and liver phospholipid are shown in Table 4.

Considerations Based on the Results Shown in Tables 3 and 4:

The following facts have been found on the basis of the results shown in Tables 3 and 4. That is, the control group has provided significantly higher values of serum total cholesterol and serum LDL-cholesterol than the untreated group and has shown a tendency of increasing serum triglyceride and serum phospholipid in comparison to the untreated group. The necrosis of hepatocytes and the balloon-like swelling in the terminal hepatic vein peripheral region of the hepatic lobule, which are observed specifically in the alcoholic hepatopathy, have been notably found in the observation of the hepatocytes through the biological microscope. The B group has shown, in comparison to the control group, a tendency of decreasing serum total cholesterol concentration, serum LDL-cholesterol concentration, serum triglyceride concentration, serum phospholipid concentration, serum ALT concentration, serum AST concentration and liver total cholesterol concentration. However, the extents approximate to the normal values thereof are small, and it has been identified that in comparison to the control group, the necrosis of hepatocytes and the balloon-like swelling in the terminal hepatic vein peripheral region of the hepatic lobule, which are specifically observed in the alcoholic hepatopathy, are slightly decreased in the observation of the hepatocytes through the biological microscope. Meanwhile, in the A group in comparison to the control group, serum total cholesterol concentration, serum LDL-cholesterol concentration, serum triglyceride concentration, serum phospholipid concentration, serum ALT concentration, serum AST concentration, liver total cholesterol concentration and liver triglyceride concentration are significantly low values, namely, values substantially equal to those of the untreated group. In the observation of the hepatocytes through the biological microscope, the necrosis of hepatocytes and the balloon-like swelling in the terminal hepatic vein peripheral region of the hepatic lobule, which are specifically observed in the alcoholic hepatopathy, have been identified to be extremely decreased in comparison to the control group.

The foregoing results have revealed that the freeze-dried powder obtained in Comparative Example 1 shows the slight activity of healing the alcoholic hepatopathy which is not suggestive of the actual use as a drug for positively healing the alcoholic hepatopathy, whereas the freeze-dried powder obtained in Example 1 shows the quite excellent activity of healing the alcoholic hepatopathy. That is, it has been found that the freeze-dried powder obtained in Example 1 (composition of the invention) has the quite excellent activity of healing the alcoholic hepatopathy and is useful as a drug.

### TEST EXAMPLE 3

With respect to the freeze-dried powder obtained in Example 1 and the freeze-dried powder obtained in Comparative Example 1, the activity of healing the alcoholic hepatopathy was evaluated by the following method different from that of Test Example 2.

An ethanol-containing liquid feed was given to 30 seven-week-old Wister-strain male rats (Nippon Charles River) for 6 days for breeding while gradually raising the ethanol content (3%→4%→5%), and these rats were successively bred with a 5% ethanol-containing liquid feed for 4 weeks. After 4 weeks of the breeding, blood was sampled from each of the rats, and the plasma was separated to measure serum lipid. These 30 rats were then divided into three groups, a control group, an A group and a B group, each group consisting of 10 rats. At this time, the 30 rats were divided so as not to cause a statistically significant difference in distribution of the average weight of the rats in each of the groups. The rats of the control group were bred by giving a 5% ethanol-containing liquid feed for 2 weeks. The rats of the A group were bred by giving for 2 weeks a liquid feed comprising a 5% ethanol-containing liquid feed and 1% of the freeze-dried powder obtained in Example 1. The rats of the B group were bred by giving for 2 weeks a liquid feed comprising a 5% ethanol-containing liquid feed and 1% of the freeze-dried powder obtained in Comparative Example 1. Further, apart from the control group, the A group and the B group, an untreated group consisting of the 10 seven-week-old Wistar-strain male rats was provided, and the rats of the untreated group were bred by giving for 6 weeks an ethanol-free liquid feed containing a mixture of maltose and dextrin in equal amounts instead of 5% ethanol for making the intake calory equal to that of the ethanol-containing liquid feed. In this case, the feeding amount (intake calory) of each liquid feed per day was limited to 70 ml (70 kcal) in the four groups. With respect to all of the four groups, on the final day of the breeding (week 6 after the start-up of the breeding), blood was sampled from the abdominal aorta of each bred rat, and the liver was extracted. After the serum was separated from the sampled blood, serum total cholesterol, serum LDL-cholesterol, serum triglyceride, serumphospholipid, serum ALT and serum AST were measured. With respect to the extracted liver, liver weight, liver total cholesterol, liver triglyceride and liver phospholipid were measured. The results obtained were shown interms of mean value±standard error (SEM), and the statistical treatment was conducted in the following manner. That is, the comparison between the untreated group and the control group was analyzed using the Student's test method, and the comparison between the control group and the A and B groups was then analyzed using the Tukey-Kramer method. In each analysis, the evaluation was conducted on condition that the level of significance of less than 0.05% was defined as significant. Further, after hepatocytes collected from the extracted liver were subjected to HE staining, morphological observation of the hepatocytes was conducted with 200× magnification using a biological microscope BX51 manufactured by Olympus Optical Co., Ltd.

The results of measuring serum total cholesterol, serum LDL-cholesterol, serumtriglyceride, serumphospholipid, serum ALT and serum AST are shown in Table 5, and the results of measuring liver weight, liver total cholesterol, liver triglyceride and liver phospholipid are shown in Table 6.

Considerations Based on the Results Shown in Tables 5 and 6:

The following facts have been found on the basis of the results shown in Tables 5 and 6. That is, the control group has shown, in comparison to the untreated group, the significantly high values with respect to serum total cholesterol concentration, serum LDL-cholesterol concentration, serum triglyceride concentration, serum phospholipid concentration, serum ALT concentration, liver weight, liver total cholesterol concentration and liver triglyceride concentration, and the necrosis of hepatocytes and the balloon-like swelling in the terminal hepatic vein peripheral region of the hepatic lobule, which are observed specifically in the alcoholic hepatopathy, have been notably found in the observation of the hepatocytes through the biological microscope.

The B group has shown, in comparison to the control group, a tendency of decreasing serum triglyceride concentration, serum total cholesterol concentration, serum phospholipid concentration, liver total cholesterol concentration and liver triglyceride concentration, but the extents approximate to the normal values thereof are small. The necrosis of hepatocytes and the balloon-like swelling in the terminal hepatic vein peripheral region of the hepatic lobule, which are observed specifically in the alcoholic hepatopathy, have been found to be slightly decreased in the observation of the hepatocytes through the biological microscope in comparison to the control group.

Meanwhile, the A group has shown, in comparison to the control group, significantly low values with respect to serum triglyceride concentration, serum total cholesterol concentration, serum phospholipid concentration, serum LDL-cholesterol concentration, liver total cholesterol concentration and liver triglyceride concentration. The necrosis of hepatocytes and the balloon-like swelling in the terminal hepatic vein peripheral region of the hepatic lobule, which are observed specifically in the alcoholic hepatopathy, have been found to be clearly decreased in the observation of the hepatocytes through the biological microscope in comparison to the control group.

The foregoing results have revealed that the freeze-dried powder obtained in Comparative Example 1 shows the slight activity of healing the alcoholic hepatopathy which is not suggestive of the actual use as a drug for positively healing the alcoholic hepatopathy, whereas the freeze-dried powder obtained in Example 1 shows the quite excellent activity of healing the alcoholic hepatopathy. That is, it has been found that the freeze-dried powder obtained in Example 1 (composition of the invention) has the quite excellent activity of healing the alcoholic hepatopathy and is useful as a drug.

As stated above in Test Example 1, it is understood that in the composition of the invention (freeze-dried powder obtained in Example 1), the liquid fraction of the barley shochu stillage has the outstanding activity of inhibiting the onset of the alcoholic hepatopathy and strongly inhibits the onset of the alcoholic hepatopathy caused by administration of ethanol. Further, as stated in Test Examples 2 and 3, it is understood that the composition of the invention (freeze-dried powder obtained in Example 1) outstandingly heals the alcoholic hepatopathy already triggered. Accordingly, it is understood that the composition of the invention has the quite excellent activity of inhibiting the onset of the alcoholic hepatopathy and the quite excellent activity of healing it and is useful as a drug.

Analysis of a Composition of Ingredients in an Unadsorbed Fraction

As will be described below, analysis of a composition of ingredients was conducted with respect to each of plural analytical samples comprising respective unadsorbed fractions which were obtained by separating plural barley shochu stillages different in lot through adsorption using an aromatic synthetic adsorbent Amberlite XAD-16 as in Example 1.

1. Production of Analytical Samples

The foregoing method [Production of barley shochu and a barley shochu stillage] was conducted plural times to prepare plural barley shochu stillages different in lot. Each of the barley shochu stillages was, in the same manner as in Example 1, centrifuged to obtain a liquid fraction of the barley shochu stillage, and 25 L of the liquid fraction and 10 L of deionized water were passed in this order through a column (resin volume 10 L) filled with a synthetic adsorbent Amberlite XAD-16 manufactured by Organo K.K. for separation by adsorption to collect an elute from the column, namely an unadsorbed fraction showing an unadsorbability to the synthetic adsorbent of the column and obtain an analytical sample made of the unadsorbed fraction. In this manner, plural analytical samples were produced.

2. Analysis of Analytical Samples

With respect to each of the plural analytical samples obtained in 1 above, a composition of amino acids, a composition of free amino acids, a composition of free saccharides, a composition of polysaccharides and a composition of organic acids constituting peptides and an average chain length of the peptides were measured. The composition of amino acids constituting the peptides was measured with an amino acid automatic analyzer (amino acid analyzer L-8500A manufactured by Hitachi Ltd.) after acid decomposition using hydrochloric acid, the composition of free amino acids with the amino acid automatic analyzer, the composition of free saccharides by HPLC (high performance liquid chromatography), the composition of polysaccharides by HPLC through hydrolysis with hydrochloric acid, the composition of organic acids by HPLC, and the average chain length of the peptides by a TNBS (2,4,6-trinitrobenzenesulfonic acid) method, respectively.

3. Results of Analysis

The results of analyzing the compositions of ingredients (based on the dry weight) of the analytical samples are shown in Table 7. As is apparent from the results shown in Table 7, it has been found that the unadsorbed fraction contains plural peptides having the average chain length of from 3.0 to 5.0, and these peptides comprise from 26 to 38% of glutamic acid, from 8 to 20% of glycine, from 6 to 10% of aspartic acid, from 6 to 9% of proline and from 5 to 8% of serine in terms of an amino acid composition ratio when the total content of amino acids derived from the peptides is defined as 100%, and that the total content of the amino acids derived from the peptides is from 9 to 14% by weight. Further, it has been found that the unadsorbed fraction contains free amino acids, free saccharides, polysaccharides and organic acids and specifically it comprises from 6 to 12% by weight of the free amino acids, from 6 to 10% by weight of the free saccharides, from 18 to 25% by weight of the polysaccharides and from 4 to 8% by weight of the organic acids. By the way, it has been found that the free amino acids have an amino acid composition comprising from 22 to 28% of proline, from 11 to 17% of alanine, from 13 to 16% of leucine, from 12 to 16% of arginine and from 15 to 20% of glutamic acid, the free saccharides have a saccharide composition comprising from 2 to 6% by weight of glucose, from 0.5 to 5% by weight of xylose and from 0.5 to 3% by weight of arabinose, and the polysaccharides have a saccharide composition comprising from 6 to 16% by weight of glucose, from 3 to 12% by weight of xylose and from 0.5 to 4% by weight of arabinose. It has been found that the organic acids include citric acid, malic acid, succinic acid and lactic acid. And, it has been found that when the unadsorbed fraction having such compositions is freeze-dried, a light yellow color is provided. By the way, since the unadsorbed fraction contains from 18 to 25% by weight of the polysaccharides as noted above, it has been presumed that some of the peptides are bound to such polysaccharides.

Moreover, the foregoing method for producing the analytical samples was conducted using the above-described aromatic synthetic adsorbents other than the synthetic adsorbent Amberlite XAD-16, namely Amberlite XAD-4, Amberlite XAD-1180 and Amberlite XAD-2000 manufactured by Organo K.K. and Sepabeads SP850 and Diaion HP20 manufactured by Mitsubishi Chemical Corp. to obtain analytical samples comprising plural unadsorbed fractions on the respective synthetic adsorbents. The resulting analytical samples were analyzed in the foregoing manner. Consequently, the results substantially equal to the results shown in Table 7 were provided.

### EXAMPLE 2

The barley shochu stillage obtained above in [Production of barley shochu and a barley shochu stillage] was centrifuged under conditions of 8,000 rpm and 10 minutes to form a liquid fraction of the barley shochu stillage, and 25 L of the liquid fraction and 10 L of deionized water were passed in this order through a column (resin volume 10 L) filled with a methacrylic synthetic adsorbent Amberlite XAD-7 manufactured by Organo K.K. to collect an unadsorbed fraction comprising a bypassed solution showing an unadsorbability to the synthetic adsorbent of the column. The unadsorbed fraction was freeze-dried using a vacuum freeze-dryer to obtain 1,060 g of a freeze-dried product. The resulting freeze-dried product was pulverized to give a light yellow powder.

### TEST EXAMPLE 4

The activity of inhibiting the onset of the alcoholic hepatopathy was evaluated in the same manner as in Test Example 1 except that the freeze-dried powder obtained in Example 2 was used instead of the freeze-dried powder obtained in Example 1. Consequently, the freeze-dried powder obtained in Example 2 showed substantially the same results as the freeze-dried powder obtained in Example 1 showed in Test Example 1.

### TEST EXAMPLE 5

The activity of healing the alcoholic hepatopathy was evaluated in the same manner as in Test Example 2 except that the freeze-dried powder obtained in Example 2 was used instead of the freeze-dried powder obtained in Example 1. Consequently, the freeze-dried powder obtained in Example 2 showed substantially the same results as the freeze-dried powder obtained in Example 1 showed in Test Example 2.

### TEST EXAMPLE 6

The activity of healing the alcoholic hepatopathy was evaluated in the same manner as in Test Example 3 except that the freeze-dried powder obtained in Example 2 was used instead of the freeze-dried powder obtained in Example 1. Consequently, the freeze-dried powder obtained in Example 2 showed substantially the same results as the freeze-dried powder obtained in Example 1 showed in Test Example 3.

As is apparent from the results shown in Test Examples 4 to 6, it is understood that in the invention, the resulting unadsorbed fractions are quite excellent in activity of inhibiting the onset of the alcoholic hepatopathy and activity of healing it in case of using the aromatic or methacrylic synthetic adsorbent.

Analysis of a Composition of Ingredients in an Unadsorbed Fraction

As will be described below, analysis of a composition of ingredients was conducted with respect to each of plural analytical samples comprising respective unadsorbed fractions which were obtained by separating plural barley shochu stillages different in lot through adsorption using a methacrylic synthetic adsorbent Amberlite XAD-7 as in Example 2.

1. Production of Analytical Samples

The foregoing method [Production of barley shochu and abarley shochu stillage] was conducted plural times to prepare plural barley shochu stillages different in lot. Each of the barley shochu stillages was, in the same manner as in Example 1, centrifuged to obtain a liquid fraction of the barley shochu stillage, and 25 L of the liquid fraction and 10 L of deionized water were passed in this order through a column (resin volume 10 L) filled with a synthetic adsorbent Amberlite XAD-7 manufactured by Organo K.K. for separation by adsorption to collect an elute from the column, namely an unadsorbed fraction showing an unadsorbability to the synthetic adsorbent of the column and obtain an analytical sample made of the unadsorbed fraction. In this manner, plural analytical samples were produced.

2. Analysis of Analytical Samples

With respect to each of the foregoing plural analytical samples, a composition of amino acids, a composition of free amino acids, a composition of free saccharides, a composition of polysaccharides and a composition of organic acids constituting peptides and an average chain length of the peptides were measured. The composition of amino acids constituting the peptides was measured with an amino acid automatic analyzer (amino acid analyzer L-8500 A manufactured by Hitachi Ltd.) after acid decomposition using hydrochloric acid, the composition of free amino acids with the amino acid automatic analyzer, the composition of free saccharides by HPLC (high performance liquid chromatography), the composition of polysaccharides by HPLC through hydrolysis with hydrochloric acid, the composition of organic acids by HPLC, and the average chain length of the peptides by a TNBS (2,4,6-trinitrobenzenesulfonic acid) method, respectively.

3. Results of Analysis

The results of analyzing the compositions of ingredients (based on the dry weight) of the analytical samples are shown in Table 8. As is apparent from the results shown in Table 8, it has been found that the unadsorbed fraction contains plural peptides having the average chain length of from 3.0 to 5.0, and these peptides comprise from 24 to 33% of glutamic acid, from 4 to 14% of glycine, from 5 to 8% of aspartic acid, from 4 to 8% of proline and from 4 to 7% of serine in terms of an amino acid composition ratio when the total content of amino acids derived from the peptides is defined as 100%, and that the total content of the amino acids derived from the peptides is from 8 to 12% by weight. Further, it has been found that the unadsorbed fraction contains free amino acids, free saccharides, polysaccharides and organic acids and specifically it comprises from 4 to 10% by weight of the free amino acids, from 5 to 8% by weight of the free saccharides, from 15 to 23% by weight of the polysaccharides and from 2 to 6% by weight of the organic acids. By the way, it has been found that the free amino acids have an amino acid composition comprising from 20 to 25% of proline, from 12 to 18% of alanine, from 11 to 17% of leucine, from 10 to 17% of arginine and from 13 to 18% of glutamic acid, the free saccharides have a saccharide composition comprising from 2 to 5% by weight of glucose, from 0.5 to 3% by weight of xylose and from 0.5 to 3% by weight of arabinose, and the polysaccharides have a saccharide composition comprising from 8 to 13% by weight of glucose, from 5 to 9% by weight of xylose and from 0.5 to 3% by weight of arabinose. It has been found that the organic acids include citric acid, malic acid, succinic acid and lactic acid. And, it has been found that when the unadsorbed fraction having such compositions is freeze-dried, a light yellow color is provided. By the way, since the unadsorbed fraction contains from 15 to 23% by weight of the polysaccharides as noted above, it has been presumed that some of the peptides are bound to such polysaccharides.

Moreover, the foregoing method for producing the analytical samples was conducted using the above-described methacrylic synthetic adsorbent other than the synthetic adsorbent Amberlite XAD-7, namely Diaion HP2MG manufactured by Mitsubishi Chemical Corp. to obtain analytical samples comprising plural unadsorbed fractions on this synthetic adsorbent. The resulting analytical samples were analyzed in the foregoing manner. Consequently, the results substantially equal to the results shown in Table 8 were provided.

### EXAMPLE 3

Water was added to the freeze-dried powder obtained in Example 1 to give a seasoning solution with the Brix degree adjusted to 30.

### COMPARATIVE EXAMPLE 3

Water was added to the freeze-dried powder obtained in Comparative Example 2 to give a seasoning solution with the Brix degree adjusted to 30.

Evaluation of the Seasoning Solutions Obtained in Example 3 and Comparative Example 3

With respect to the seasoning solutions obtained in Example 3 and Comparative Example 3, the organoleptic examination was performed by 12 panelists. The results of the organoleptic examination are shown in Table 9. As is apparent from the results shown in Table 9, it has been found that the seasoning solution obtained in Example 3, in comparison to the seasoning solution obtained in Comparative Example 3, shows a good palatability with bitterness and an acrid taste ascribable to the barley shochu stillage extremely reduced as well as a low degree of coloration.

### EXAMPLE 4

The freeze-dried powder obtained in Example 1 was mixed with other food materials according to the following formulation to prepare a dressing.

Formulation: 25% by weight of vegetable oil, 10% by weight of the freeze-dried powder obtained in Example 1, 10% by weight of fermented vinegar, 22% by weight of soy sauce, 20% by weight of onion, 10% by weight of sugar, and 3% by weight of lemon juice.

### COMPARATIVE EXAMPLE 4

A dressing was prepared as in Example 4 except that the freeze-dried powder obtained in Comparative Example 2 was used instead of the freeze-dried powder obtained in Example 1.

Evaluation of the Dressings Obtained in Example 4 and Comparative Example 4

With respect to the dressings obtained in Example 4 and Comparative Example 4, the organoleptic examination was performed by 12 panelists. The results of the organoleptic examination are shown in Table 10. As is apparent from the results shown in Table 10, it has been found that the dressing obtained in Example 4, in comparison to the dressing obtained in Comparative Example 4, has no doubt a rich taste and is also excellent in color.

### EXAMPLE 5

The freeze-dried powder obtained in Example 1 was mixed with other food materials according to the following formulation to prepare a health drink.

Formulation: 10% by weight of lemon juice, 10% by weight of the freeze-dried powder obtained in Example 1, 10% by weight of honey, 5% by weight of a fructose/sucrose solution, and 65% by weight of water.

### COMPARATIVE EXAMPLE 5

A health drink was prepared as in Example 5 except that the freeze-dried powder obtained in Comparative Example 2 was used instead of the freeze-dried powder obtained in Example 1.

Evaluation of the Health Drinks Obtained in Example 5 and Comparative Example 5

With respect to the health drinks obtained in Example 5 and Comparative Example 5, the organoleptic examination was performed by 12 panelists. Consequently, it has been found that the health drink obtained in Example 5, in comparison to the health drink obtained in Comparative Example 5, has a rich taste, is easy to drink without an odd taste, and is excellent in color.

### EXAMPLE 6

The freeze-dried powder obtained in Example 1 was mixed with other food materials according to the following formulation to make bread.

Formulation: 47% by weight of a powder with a high stickiness, 2% by weight of the freeze-dried powder obtained in Example 1, 2% by weight of butter, 4% by weight of sugar, 1.5% by weight of skimmilk, 1% by weight of table salt, 42% by weight of water, and 0.5% by weight of yeast

### COMPARATIVE EXAMPLE 6

Bread was made as in Example 6 except that the freeze-dried powder obtained in Comparative Example 2 was used instead of the freeze-dried powder obtained in Example 1.

Evaluation of Breads Obtained in Example 6 and Comparative Example 6

With respect to the breads obtained in Example 6 and Comparative Example 6, the organoleptic examination was performed by 12 panelists. Consequently, it has been found that the bread obtained in Example 6, in comparison to the bread obtained in Comparative Example 6, has not only a rich taste but also a resilient feel in the mouth, and is not problematic in color.

The foregoing results of Examples 3 to 6 have revealed that the freeze-dried powder obtained in Example 1 (composition of the invention) has the quite excellent palatability which is superior to the palatability provided by the freeze-dried powder in Comparative Example 2.

As is apparent from the foregoing detailed description, according to the invention, the composition which is quite excellent in activity of inhibiting the onset of the alcoholic hepatopathy and activity of healing it and is useful as a drug and the food composition which is quite excellent in activity of inhibiting the onset of the alcoholic hepatopathy and activity of healing it and has the quite excellent palatability can be obtained from the barley shochu stillage byproduced in the production of shochu from a barley as a raw material.

TABLE 1

| | Total cholesterol | HDL-cholesterol | LDL-cholesterol | Triglyceride | Phospholipid | Free fatty acid | ALT |
|---|---|---|---|---|---|---|---|
| Test group | (mg/dl) | (mg/dl) | (mg/dl) | (mg/dl) | (mg/dl) | (μEq/l) | (U/I) |
| Untreated group | 68 ± 3 | 34 ± 2 | 10 ± 1 | 93 ± 22 | 147 ± 7 | 455 ± 34 | 31 ± 3 |
| Control group | 101 ± 7*** | 46 ± 4** | 21 ± 3*** | 219 ± 38*** | 209 ± 12*** | 518 ± 85 | 43 ± 3* |
| A group | 105 ± 3 | 53 ± 2 | 13 ± 1^{##} | 84 ± 12^{##} | 215 ± 7 | 801 ± 221 | 33 ± 4^{#} |
| (Example 1) | | | | | | | |
| B group | 125 ± 6 | 58 ± 3 | 17 ± 1 | 147 ± 28 | 238 ± 18 | 776 ± 145 | 40 ± 3 |
| (Comparative | | | | | | | |
| Example 1) | | | | | | | |
| C group | 126 ± 7 | 55 ± 3 | 17 ± 1 | 153 ± 24 | 231 ± 14 | 769 ± 151 | 41 ± 4 |
| (Comparative | | | | | | | |
| Example 2) | | | | | | | |
| D group | 129 ± 8 | 60 ± 3 | 17 ± 1 | 128 ± 27 | 228 ± 8 | 626 ± 55 | 47 ± 4^{##} |
| (Reference | | | | | | | |
| Example 1) | | | | | | | |

(Mean value ± SEM)
*p < 0.05,
**p < 0.01,
***p < 0.001 (Student's test) in comparison to the untreated group
#p < 0.05,
##p < 0.01 (Tukey-Kramer) in comparison to the control group

TABLE 2

| Test group | Liver weight (g) | Total cholesterol (mg/dl) | Triglyceride (mg/dl) | Phospholipid (mg/dl) |
|---|---|---|---|---|
| Untreated group | 12 ± 1 | 3.3 ± 0.1 | 19.7 ± 1.9 | 4.5 ± 0.6 |
| Control group | 13 ± 1 | 3.3 ± 0.2 | 68.2 ± 3.7*** | 18.7 ± 1.2*** |
| A group | 13 ± 1 | 4.2 ± 0.2 | 46.4 ± 3.8^{##} | 12.8 ± 1.7 |
| (Example 1) | | | | |
| B group | 13 ± 1 | 3.6 ± 0.2 | 64.0 ± 4.8 | 19.0 ± 2.2 |
| (Comparative Example 1) | | | | |
| C group | 13 ± 1 | 3.4 ± 0.2 | 64.4 ± 3.5 | 18.7 ± 1.3 |
| (Comparative Example 2) | | | | |
| D group | 17 ± 1 | 5.0 ± 0.3 | 78.2 ± 6.4 | 20.7 ± 2.7^{##} |
| (Reference Example 1) | | | | |

(Mean value ± SEM)
***p < 0.001 (Student's test) in comparison to the untreated group
^{##}p < 0.01 (Tukey-Kramer) in comparison to the control group

TABLE 3

| | Total cholesterol | LDL-cholesterol | Triglyceride | Phospholipid | | |
|---|---|---|---|---|---|---|
| Test group | (mg/dl) | (mg/dl) | (mg/dl) | (mg/dl) | ALT (U/I) | AST (U/I) |
| Untreated group | 71.4 ± 3.0 | 9.2 ± 0.9 | 37.6 ± 7.0 | 125.0 ± 4.9 | 33.2 ± 1.6 | 76.9 ± 6.0 |
| Control group | 82.1 ± 3.8* | 14.5 ± 1.3* | 61.8 ± 6.3 | 139.2 ± 5.8 | 47.4 ± 11.9 | 92.2 ± 5.1* |
| A group (Example 1) | 69.1 ± 4.5^{#} | 9.5 ± 1.0^{#} | 33.8 ± 6.7^{#} | 116.3 ± 6.5^{##} | 30.0 ± 1.2^{#} | 73.5 ± 2.2^{#} |
| B group | 77.9 ± 2.4 | 14.6 ± 0.6 | 58.2 ± 4.9 | 136.4 ± 4.4 | 44.8 ± 1.5 | 88.1 ± 6.6 |
| (Comparative Example 1) | | | | | | |

(Mean value ± SEM)
*p < 0.05,
**p < 0.01,
***p < 0.001 (Student's test) in comparison to the untreated group
#p < 0.05,
##p < 0.01 (Tukey-Kramer) in comparison to the control group

TABLE 4

| Test group | Liver weight (g) | Total cholesterol (mg/dl) | Triglyceride (mg/dl) | Phospholipid (mg/dl) |
|---|---|---|---|---|
| Untreated group | 2.42 ± 0.06 | 3.53 ± 0.33 | 45.42 ± 1.85 | 55.64 ± 2.21 |
| Control group | 2.47 ± 0.04 | 7.79 ± 1.43 | 54.62 ± 6.33 | 53.28 ± 4.36 |
| A group | 2.38 ± 0.05 | 3.91 ± 0.83^{#} | 31.19 ± 5.82^{##} | 51.72 ± 3.08 |
| (Example 1) | | | | |
| B group | 2.48 ± 0.03 | 6.34 ± 0.44 | 50.19 ± 4.00 | 52.65 ± 3.15 |
| (Comparative Example 1) | | | | |

(Mean value ± SEM)
*: p < 0.05,
**: p < 0.01,
***: p < 0.001 (Student's test) in comparison to the untreated group
^{#}p < 0.05,
^{##}p < 0.01 (Tukey-Kramer) in comparison to the control group

TABLE 5

| | Total cholesterol | LDL-cholesterol | Triglyceride | Phospholipid | | |
|---|---|---|---|---|---|---|
| Test group | (mg/dl) | (mg/dl) | (mg/dl) | (mg/dl) | ALT (U/I) | AST (U/I) |
| Untreated group | 71.4 ± 3.0 | 9.2 ± 0.9 | 37.6 ± 7.0 | 125.0 ± 4.9 | 33.2 ± 1.6 | 86.9 ± 6.0 |
| Control group | 132.5 ± 4.8*** | 14.5 ± 1.3*** | 110.8 ± 22.2** | 223.9 ± 8.4*** | 61.9 ± 5.4** | 113.8 ± 5.6 |
| A group | 105.1 ± 4.1^{##} | 11.0 ± 1.1^{#} | 48.9 ± 9.1^{##} | 163.5 ± 7.7^{##} | 53.4 ± 3.2 | 98.1 ± 12.2^{#} |
| (Example 1) | | | | | | |
| B group | 125.4 ± 6.4 | 14.6 ± 0.6 | 87.6 ± 11.8 | 205.1 ± 7.1 | 60.3 ± 7.7 | 103.6 ± 12.8 |
| (Comparative Example 1) | | | | | | |

(Mean value ± SEM)
*p < 0.05,
**p < 0.01,
***p < 0.001 (Student's test) in comparison to the untreated group
#p < 0.05,
##p < 0.01 (Tukey-Kramer) in comparison to the control group

TABLE 6

| Test group | Liver weight (g) | Total cholesterol (mg/dl) | Triglyceride (mg/dl) | Phospholipid (mg/dl) |
|---|---|---|---|---|
| Untreated group | 2.42 ± 0.06 | 3.53 ± 0.33 | 45.42 ± 1.85 | 55.64 ± 2.21 |
| Control group | 3.28 ± 0.09*** | 10.42 ± 1.63*** | 122.82 ± 11.71*** | 63.59 ± 4.90 |
| A group | 3.15 ± 0.15 | 4.36 ± 0.61^{###} | 71.28 ± 7.75^{###} | 61.08 ± 3.08 |
| (Example 1) | | | | |
| B group | 3.09 ± 0.15 | 9.45 ± 0.50 | 118.70 ± 5.04 | 60.32 ± 4.68 |
| (Comparative Example 1) | | | | |

(Mean value ± SEM)
*: p < 0.05,
**: p < 0.01,
***p < 0.001 (Student's test) in comparison to the untreated group
#: p < 0.05,
##: p < 0.01,
^{###}p < 0.001 (Tukey-Kramer) in comparison to the control group

TABLE 7

| Peptides | Average chain length | | 3.0 to 5.0 |
|---|---|---|---|
| | Content of constituent amino acids (wt. %) | | 9 to 14 |
| | Composition ratio | glutamic acid (%) | 26 to 38 |
| | | glycine (%) | 8 to 20 |
| | | aspartic acid (%) | 6 to 10 |
| | | proline (%) | 6 to 9 |
| | | serine (%) | 5 to 8 |
| Free amino acids | Free amino acids (wt. %) | | 6 to 12 |
| | Composition ratio | proline (%) | 22 to 28 |
| | | alanine (%) | 11 to 17 |
| | | leucine (%) | 13 to 16 |
| | | arginine (%) | 12 to 16 |
| | | glutamic acid (%) | 15 to 20 |

| | |
|---|---|
| Free saccharides (wt. %) | 6 to 10 |
| glucose (wt. %) | 2 to 6 |
| xylose (wt. %) | 0.5 to 5 |
| arabinose (wt. %) | 0.5 to 3 |
| Polysaccharides (wt. %) | 18 to 25 |
| glucose (wt. %) | 6 to 16 |
| xylose (wt. %) | 3 to 12 |
| arabinose (wt. %) | 0.5 to 4 |
| Organic acids (wt. %) | 4 to 8 |

TABLE 8

| Peptides | Average chain length | | 3.0 to 5.0 |
|---|---|---|---|
| | Content of constituent amino acids (wt. %) | | 8 to 12 |
| | Composition ratio | glutamic acid (%) | 24 to 33 |
| | | glycine (%) | 4 to 14 |
| | | aspartic acid (%) | 5 to 8 |
| | | proline (%) | 4 to 8 |
| | | serine (%) | 4 to 7 |
| Free amino acids | Free amino acids (wt. %) | | 4 to 10 |
| | Composition ratio | proline (%) | 20 to 25 |
| | | alanine (%) | 12 to 18 |
| | | leucine (%) | 11 to 17 |
| | | arginine (%) | 10 to 17 |
| | | glutamic acid (%) | 13 to 18 |

| | |
|---|---|
| Free saccharides (wt. %) | 5 to 8 |
| glucose (wt. %) | 2 to 5 |
| xylose (wt. %) | 0.5 to 3 |
| arabinose (wt. %) | 0.5 to 3 |
| Polysaccharides (wt. %) | 15 to 23 |
| glucose (wt. %) | 8 to 13 |
| xylose (wt. %) | 5 to 9 |
| arabinose (wt. %) | 0.5 to 3 |
| Organic acids (wt. %) | 2 to 6 |

TABLE 9

Organoleptic evaluation

| Panelist | Example 3 | Comparative Example 3 |
|---|---|---|
| A | 3 | 3 |
| B | 2 | 4 |
| C | 2 | 2 |
| D | 3 | 1 |
| E | 1 | 2 |
| F | 2 | 3 |
| G | 1 | 3 |
| H | 1 | 2 |
| I | 3 | 2 |
| J | 2 | 3 |
| K | 1 | 4 |
| L | 1 | 2 |

Criteria of organoleptic evaluation on a taste of a seasoning solution
1: good
2: slightly good
3: slightly bad
4: bad

TABLE 10

Organoleptic evaluation

| Panelist | Example 4 | Comparative Example 4 |
|---|---|---|
| A | 3 | 2 |
| B | 1 | 4 |
| C | 3 | 3 |
| D | 1 | 4 |
| E | 2 | 2 |
| F | 2 | 4 |
| G | 2 | 1 |
| H | 1 | 3 |
| I | 1 | 2 |
| J | 3 | 2 |
| K | 2 | 4 |
| L | 3 | 3 |

Criteria of organoleptic evaluation on a taste of a dressing
1: good
2: slightly good
3: slightly bad
4: bad

1. A composition having an activity of inhibiting the onset of alcoholic hepatopathy and an activity of healing it, the composition comprising an unadsorbed fraction which is formed by subjecting a barley shochu stillage byproduced in the production of shochu from a barley as a raw material to solid-liquid separation to obtain a liquid fraction and subjecting the liquid fraction to a separation treatment by adsorption using a synthetic adsorbent, in which the unadsorbed fraction contains plural peptides having an average chain length of from 3.0 to 5.0, and these peptides comprise from 24 to 38% of glutamic acid, from 4 to 20% of glycine, from 5 to 10% of aspartic acid, from 4 to 9% of proline and from 4 to 8% of serine in terms of an amino acid composition ratio when the total content of amino acids derived from the peptides is defined as 100%.

2. The composition according to claim 1, wherein the total content of amino acids derived from the peptides is from 8 to 14% by weight.

3. The composition according to claim 1, wherein the unadsorbed fraction further contains free amino acids, free saccharides, polysaccharides and organic acids.

4. The composition according to claim 3, wherein the unadsorbed fraction contains from 4 to 12% by weight of the free amino acids, from 5 to 10% by weight of the free saccharides, from 15 to 25% by weight of the polysaccharides and from 2 to 8% by weight of the organic acids.

5. The composition according to claim 1, wherein the unadsorbed fraction is in the form of a freeze-dried powder.

6. The composition according to claim 1, which is used as a drug.

7. The composition according to claim 1, wherein the synthetic adsorbent is an aromatic synthetic adsorbent or a methacrylic synthetic adsorbent.

8. A process for producing a composition having an activity of inhibiting the onset of alcoholic hepatopathy and an activity of healing it and comprising an unadsorbed fraction, which comprises a step of subjecting a barley shochu stillage byproduced in the production of shochu from a barley as a raw material to solid-liquid separation to obtain a liquid fraction and a step of subjecting the liquid fraction to a separation treatment by adsorption using a synthetic adsorbent to obtain the unadsorbed fraction, in which the unadsorbed fraction contains plural peptides having an average chain length of from 3.0 to 5.0, and these peptides comprise from 24 to 38% of glutamic acid, from 4 to 20% of glycine, from 5 to 10% of aspartic acid, from 4 to 9% of proline and from 4 to 8% of serine in terms of an amino acid composition ratio when the total content of amino acids derived from the peptides is defined as 100%.

9. The process according to claim 8, which further comprises a step of freeze-drying the unadsorbed fraction.

10. The process according to claim 8, wherein the synthetic adsorbent is an aromatic synthetic adsorbent or a methacrylic synthetic adsorbent.

11. A process for producing a barley shochu and a composition comprising an unadsorbed fraction, characterized by comprising a step (A) of fermenting a barley koji produced from a husked barley or a polished barley as a raw material and a shochu yeast to form a mature mash and distilling the mature mash to produce the barley shochu, and a step (B) of subjecting a barley shochu stillage byproduced in the production of the barley shochu in the step (A) to solid-liquid separation to obtain a liquid fraction and subjecting the liquid fraction to a separation treatment by adsorption using a synthetic adsorbent to obtain the unadsorbed fraction, in which the unadsorbed fraction contains plural peptides having an average chain length of from 3.0 to 5.0, these peptides comprise from 24 to 38% of glutamic acid, from 4 to 20% of glycine, from 5 to 10% of aspartic acid, from 4 to 9% of proline and from 4 to 8% of serine in terms of an amino acid composition ratio when the total content of amino acids derived from the peptides is defined as 100%, an activity of inhibiting the onset of alcoholic hepatopathy and an activity of healing it are provided, and the steps (A) and (B) are conducted continuously.

12. The process according to claim 11, characterized in that in the step (A), a husked barley or a polished barley prepared separately is fermented along with the barley koji and the shochu yeast in obtaining the mature mash.

13. The process according to claim 11 or 12, wherein the synthetic adsorbent is an aromatic synthetic adsorbent or a methacrylic synthetic adsorbent.

14. A food composition having an activity of inhibiting the onset of alcoholic hepatopathy and an activity of healing it as well as an excellent palatability, the composition comprising an unadsorbed fraction which is formed by subjecting a barley shochu stillage byproduced in the production of shochu from a barley as a raw material to solid-liquid separation to obtain a liquid fraction and subjecting the liquid fraction to a separation treatment by adsorption using a synthetic adsorbent, in which the unadsorbed fraction contains plural peptides having an average chain length of from 3.0 to 5.0, and these peptides comprise from 24 to 38% of glutamic acid, from 4 to 20% of glycine, from 5 to 10% of aspartic acid, from 4 to 9% of proline and from 4 to 8% of serine in terms of an amino acid composition ratio when the total content of amino acids derived from the peptides is defined as 100%.

15. The food composition according to claim 14, wherein the total content of amino acids derived from the peptides is from 8 to 14% by weight.

16. The food composition according to claim 14, wherein the unadsorbed fraction further contains free amino acids, free saccharides, polysaccharides and organic acids.

17. The food composition according to claim 16, wherein the unadsorbed fraction contains from 4 to 12% by weight of the free amino acids, from 5 to 10% by weight of the free saccharides, from 15 to 25% by weight of the polysaccharides and from 2 to 8% by weight of the organic acids.

18. The food composition according to claim 14, wherein the unadsorbed fraction is in the form of a freeze-dried powder.

19. The food composition according to claim 14, which is used as a seasoning.

20. The food composition according to claim 14, wherein the synthetic adsorbent is an aromatic synthetic adsorbent or a methacrylic synthetic adsorbent.

21. A process for producing a food composition comprising an unadsorbed fraction, which comprises a step of subjecting a barley shochu stillage byproduced in the production of shochu from a barley as a raw material to solid-liquid separation to obtain a liquid fraction and a step of subjecting the liquid fraction to a separation treatment by adsorption using a synthetic adsorbent to obtain the unadsorbed fraction, in which the unadsorbed fraction contains plural peptides having an average chain length of from 3.0 to 5.0, these peptides comprise from 24 to 38% of glutamic acid, from 4 to 20% of glycine, from 5 to 10% of aspartic acid, from 4 to 9% of proline and from 4 to 8% of serine in terms of an amino acid composition ratio when the total content of amino acids derived from the peptides is defined as 100%.

22. The process according to claim 21, which further comprises a step of freeze-drying the unadsorbed fraction.

23. The process according to claim 21, wherein the synthetic adsorbent is an aromatic synthetic adsorbent or a methacrylic synthetic adsorbent.

24. A process for continuously producing a barley shochu and food composition comprising an unadsorbed fraction, characterized by comprising a step (A) of fermenting a barley koji produced from a husked barley or a polished barley as a raw material and a shochu yeast to form a mature mash and distilling the mature mash to produce the barley shochu, and a step (B) of subjecting a barley shochu stillage byproduced in the production of the barley shochu in the step (A) to solid-liquid separation to obtain a liquid fraction and subjecting the liquid fraction to a separation treatment by adsorption using a synthetic adsorbent to obtain the unadsorbed fraction, in which the unadsorbed fraction contains plural peptides having an average chain length of from 3.0 to 5.0, these peptides comprise from 24 to 38% of glutamic acid, from 4 to 20% of glycine, from 5 to 10% of aspartic acid, from 4 to 9% of proline and from 4 to 8% of serine in terms of an amino acid composition ratio when the total content of amino acids derived from the peptides is defined as 100%, an activity of inhibiting the onset of alcoholic hepatopathy, an activity of healing it and an excellent palatability are provided, and the steps (A) and (B) are conducted continuously.

25. The process according to claim 24, characterized in that in the step (A), a husked barley or a polished barley prepared separately is fermented along with the barley koji and the shochu yeast in obtaining the mature mash.

26. The process according to claim 24 or 25, wherein the synthetic adsorbent is an aromatic synthetic adsorbent or a methacrylic synthetic adsorbent.
